SEQUENCE LISTING (13 sequences)

SEQ ID NO: 1 - DNA, 2440 bp, synthetic construct
  source 1..2440: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgagccaccatggacatgagggtccctgctcagctcctggggctcctgctgctctggctctcaggtgccagatgtgatgccagcctgttcagcggccggatcctgatcaggaacaacagcgaccaggacgagctggacaccgaggccgaagtgagcaggaggctggagaacagactggtgctgctgttctttggcgccggagcctgccctcagtgccaggccttcgtgcccatcctgaaggatttctttgtgcggctgaccgacgagttctacgtgctgagagccgcccagctggccctggtgtatgtgagccaggacagcaccgaggagcagcaggacctgttcctgaaggacatgcccaagaagtggctgttcctgcccttcgaggacgacctgcggagatgagctagcaccggttgtacaagtcaagcggccaaccctccctagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - protein, 131 aa, synthetic construct
MDMRVPAQLLGLLLLWLSGARCDASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 3 - DNA, 3121 bp, synthetic construct
  source 1..3121: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgagccaccatggacatgagggtccctgctcagctcctggggctcctgctgctctggctctcaggtgccagatgtgatgccagcctgttcagcggccggatcctgatcaggaacaacagcgaccaggacgagctggacaccgaggccgaagtgagcaggaggctggagaacagactggtgctgctgttctttggcgccggagcctgccctcagtgccaggccttcgtgcccatcctgaaggatttctttgtgcggctgaccgacgagttctacgtgctgagagccgcccagctggccctggtgtatgtgagccaggacagcaccgaggagcagcaggacctgttcctgaaggacatgcccaagaagtggctgttcctgcccttcgaggacgacctgcggagaggacgacccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataatgagctagcaccggttgtacaagtcaagcggccaaccctccctagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 4 - protein, 357 aa, synthetic construct
MDMRVPAQLLGLLLLWLSGARCDASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRGRPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 23 aa, Homo sapiens
MDMRVPAQLLGLLLLWLSGARCD

SEQ ID NO: 6 - protein, 108 aa, Homo sapiens
ASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 7 - protein, 226 aa, Homo sapiens
GRPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 22 aa, Homo sapiens
MDMRVPAQLLGLLLLWLSGARC

SEQ ID NO: 9 - protein, 23 aa, Homo sapiens
METDTLLWVLLLWVPGSTGDDIV

SEQ ID NO: 10 - protein, 131 aa, synthetic construct
METDTLLWVLLLWVPGSTGDDIVASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 11 - protein, 130 aa, synthetic construct
MDMRVPAQLLGLLLLWLSGARCASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRR

SEQ ID NO: 12 - protein, 357 aa, synthetic construct
METDTLLWVLLLWVPGSTGDDIVASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRGRPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 356 aa, synthetic construct
MDMRVPAQLLGLLLLWLSGARCASLFSGRILIRNNSDQDELDTEAEVSRRLENRLVLLFFGAGACPQCQAFVPILKDFFVRLTDEFYVLRAAQLALVYVSQDSTEEQQDLFLKDMPKKWLFLPFEDDLRRGRPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0091133 A1
(43) Pub. Date: Apr. 2, 2026

(54) ROD-DERIVED CONE VIABILITY FACTOR FUSION PROTEIN

(71) Applicant: PHARMA CINQ, LLC, Rockville, MD (US)

(72) Inventors: Tianci LUO, Fulton, MD (US); Daniel LIPUT, Olney, MD (US); Fong Qi LIANG, Mount Airy, MD (US)

(21) Appl. No.: 19/339,969

(22) Filed: Sep. 25, 2025

Related U.S. Application Data
(60) Provisional application No. 63/700,312, filed on Sep. 27, 2024.
(60) Provisional application No. 63/702,584, filed on Oct. 2, 2024.

(51) Int. Cl. A61K 48/00; A61K 38/00; A61P 27/02; C07K 14/47; C12N 15/62; C12N 15/86

(52) U.S. Cl. CPC A61K 48/005; A61K 48/0075; A61P 27/02; C07K 14/4702; C12N 15/62; C12N 15/86; A61K 38/00; C07K 2319/02; C07K 2319/30; C12N 2750/14143

(57) ABSTRACT

The present invention relates to a fusion protein comprising a) a short rod-derived cone viability factor (RdCVF-S), b) a hydrophilic peptide, and c) a signal peptide, along with the nucleic acids encoding those peptides and viral vectors containing these nucleic acids. The invention also relates to compositions and pharmaceutical preparations comprising these nucleic acids or vectors, methods of producing or secreting the fusion protein comprising RdCVF-S protein, an immunoglobulin constant region, and a human signal sequence, and methods of treatment.

### CROSS-REFERENCE TO ELECTRONIC SEQUENCE LISTING

The application contains a Sequence Listing which has been submitted electronically in .XML format and is hereby incorporated by reference in its entirety. Said .XML copy, created on Sep. 23, 2025, is named “0288-0068US1.xml” and is 19,437 bytes in size. The sequence listing contained in this .XML file is part of the specification and is hereby incorporated by reference herein in its entirety.

### FIELD OF THE INVENTION

The present disclosure provides a fusion protein comprising a Rod-derived cone viability factor (RdCVF) that is fused to a secretory signal and an immunoglobulin constant region, resulting in an enhanced expression and secretion of the RdCVF protein. The disclosure further relates to methods of producing the RdCVF fusion protein, delivery vehicles of the fusion protein, and potential methods for treating eye diseases using the fusion protein.

### BACKGROUND

Rod-derived cone viability factor (RdCVF) is a thioredoxin-like protein specifically expressed by rod photoreceptor cells in the retina (Léveillard et al. (2004) Nature Genetics 36:755-759 and the supplemental information). Two different RdCVF genes are found in humans and they are designated RdCVF1 and RdCVF2. Both RdCVF genes encode two products via alternative splicing: a full-length protein and a C-terminal post-transcriptionally truncated protein, known as RdCVF-long and RdCVF-short, respectively. RdCVF1-short is described as a secreted trophic factor for promoting cone survival, and RdCVF1-Long as a redox-active enzyme that interacts with intracellular proteins (Léveillard et al. (2010) Sci Transl Med. 2(26): 26ps16). For example, tau is described as a binding partner for RdCVF1-L and tau is exclusively intracellular (Fridlich et al. (2009) Molecular & Cellular Proteomics 8(6):1206-18).

RdCVF has great potential as a candidate for treating eye diseases including inherited retina diseases such as retinitis pigmentosa (RP) and geographic atrophy (GA). Unfortunately, expressing and secreting RdCVF presents a major challenge in designing therapies that promote cone cell survival, which may be due to the hydrophobicity of the amino acid composition in RdCVF proteins. Therefore, this disclosure provides a fusion protein that increases the hydrophilicity index of RdCVF proteins, improving the expression and secretion of RdCVF proteins in potential therapies for eye diseases.

Citation or discussion of a reference herein shall not be construed as an admission that such is prior art to the present invention.

### SUMMARY OF THE INVENTION

In some embodiments, the disclosure provides a fusion protein comprising a first N-terminal signal peptide, a second peptide C-terminal to the signal peptide, and a third peptide C-terminal to the second peptide, wherein the second peptide or the third peptide is an RdCVF-short peptide and the other is an immunoglobulin constant region.

In some embodiments, the disclosure provides a nucleotide sequence encoding a fusion protein comprising a first N-terminal signal peptide, a second peptide C-terminal to the signal peptide, and a third peptide C-terminal to the second peptide sequence, wherein the second peptide or the third peptide sequence is an RdCVF-short peptide sequence and the other is an immunoglobulin constant region sequence.

In some embodiments, the disclosure provides a fusion protein comprising a first N-terminal signal peptide, a second peptide C-terminal to the signal peptide, and a third peptide C-terminal to the second peptide, wherein the first peptide is a human immunoglobulin kappa chain (Igk) signal peptide, the second peptide or the third peptide is an RdCVF-short peptide and the other is a hydrophilic peptide.

In some embodiments, the disclosure provides a nucleotide sequence encoding a fusion protein comprising a first N-terminal signal peptide, a second peptide C-terminal to the signal peptide, and a third peptide C-terminal to the second peptide sequence, wherein the first peptide is a human immunoglobulin kappa chain (Igk) signal sequence, the second peptide or the third peptide sequence is an RdCVF-short peptide sequence and the other is a hydrophilic peptide.

In some embodiments, the disclosure relates to an expression vector comprising a nucleic acid of this disclosure, operatively linked to a control sequence.

In some embodiments, the disclosure relates to a cell comprising a fusion protein, a nucleic acid, or an expression vector of this disclosure.

In some embodiments, the disclosure relates to a pharmaceutical composition comprising (i) a fusion protein, a nucleic acid, an expression vector, and/or a cell of this disclosure and (ii) a pharmaceutically acceptable carrier.

In some embodiments, the disclosure relates to a method of treating a mammalian subject in need thereof, comprising administering to the subject an effective amount of a fusion protein, a nucleic acid, a vectors, a cell, and/or a pharmaceutical composition of this disclosure, thereby treating the condition in the subject.

In some embodiments, the disclosure relates to a method of protecting ocular photoreceptor cells in a subject in need thereof, comprising administering to the eye of the subject an effective amount of a fusion protein, a nucleic acid, a vectors, a cell, and/or a pharmaceutical composition of this disclosure, thereby protecting the ocular photoreceptor cells in the subject.

### BRIEF DESCRIPTION OF THE DRAWINGS

The following drawings form part of the present specification and are included to further demonstrate exemplary embodiments of certain aspects of the present disclosure.

FIG. 1 shows an SDS-PAGE silver stain analysis of purified recombinant rAAV8.hIgk-hRdCVF1S and rAAV8.hIgk-hRdCVF1S.hIgG4-Fc vector particles. The presence of AAV particles was confirmed by visualization of the VP1, VP2 and VP3 capsid proteins.

FIG. 2 shows Western blot analysis of hRdCVF1S expression and secretion in rAAV8.hIgk-hRdCVF1S and rAAV8.hIgk-hRdCVF1S.hIgG4-Fc vector transduced human HEK293 cells. Left Panel: A protein immunoreactive to anti-RdCVF antibodies was detected at approximately 12 KDa (near the theoretical molecular weight of hRdCVF1S) in cell lysate transduced with rAAV8.hIgk-hRdCVF1S vector (pointed by arrow). A protein immunoreactive to anti-RdCVF antibodies was detected at approximately 38 KDa (near the theoretical molecular weight of hRdCVF1S.hIgG4-Fc) in cell lysate transduced with rAAV8.hIgk-hRdCVF1S.hIgG4-Fc vector (pointed by arrow). Right Panel: A protein immunoreactive to anti-RdCVF antibodies was detected at approximately 38 KDa (near the theoretical molecular weight of hRdCVF1S.hIgG4-Fc) in cell culture media transduced with rAAV8.hIgk-hRdCVF1S.hIgG4-Fc vector (pointed by arrow).

### DETAILED DESCRIPTION

It should be understood that any embodiment described herein, including those described only in the examples, can be combined with any one or more other embodiments, unless such combination is expressly disclaimed or is improper. Thus, the term “embodiment”, as used herein, is not to be considered as excluding features recited in other embodiments.

Unless otherwise defined herein, scientific and technical terms used in the present disclosure shall have meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or “about” can mean rounded to the nearest significant digit.

As used herein, “between” is a range inclusive of the ends of the range. For example, a number between x and y explicitly includes the numbers x and y and any numbers that fall within x and y.

The terms “optional” or “optionally” as used herein refer to a feature or structure being present or not, or an event or circumstance occurring or not, and that the description includes instances in which a particular feature or structure is present and instances in which the feature or structure is absent, or instances in which the event or circumstance occurs and instances in which the event or circumstance does not occur.

The term “protein” as used herein refers to a polymeric form of amino acids of any length, which can include coded and non-coded amino acids, chemically or biochemically modified or derivatized amino acids, and polypeptides having modified peptide backbones.

As used herein, in embodiments, the term “fusion protein” refers to a protein consisting of at least two domains that are encoded by separate genes that have been joined so that they are transcribed and translated as a single unit, producing a single polypeptide. The translation of the fusion genes result in a single or multiple polypeptides with functional properties derived from each of the original proteins. Use of the term “fusion protein” is meant to include chimeric proteins, fusion genes, recombinant fusion proteins, and fusion protein-related polypeptides, as long as they exhibit the desired biologically activity or function.

The term “recombinant” is a common term and known and understood in the art. When referring to a nucleic acid (e.g., a gene), the recombinant can be used, for example, to describe a nucleic acid that has been removed from its naturally occurring environment, a nucleic acid that is not associated with all or a portion of a nucleic acid abutting or proximal to the nucleic acid when it is found in nature, a nucleic acid that is operatively linked to a nucleic acid that it is not linked to in nature, or a nucleic acid that does not occur in nature. The term “recombinant” can be used, e.g., to describe cloned DNA isolates, or a nucleic acid including a chemically-synthesized nucleotide analog. A nucleic acid is also considered “recombinant” if it contains any modifications that do not naturally occur to the corresponding nucleic acid in a genome. For instance, an endogenous coding sequence is considered “recombinant” if it contains an insertion, deletion, or a point mutation introduced artificially, e.g., by human intervention. A “recombinant nucleic acid” also includes a nucleic acid integrated into a host cell chromosome at a heterologous site and a nucleic acid construct present as an episome. When “recombinant” is used to describe a protein, it can refer to, for example, a protein that is produced in a cell of a different species or type as compared to the species or type of cell that produces the protein in nature. The term “recombinant host cell” as used herein refers to a cell into which a recombinant nucleic acid has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “vector” as used herein refers to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome.

The term “operatively linked” (or “operably linked”) as used herein means, with reference to a juxtaposition of two or more components (such as sequence elements), that the components are arranged such that both components function normally and allow the possibility that at least one of the components can mediate a function that is exerted upon at least one of the other components. For example, a nucleic acid that is operatively linked to a promoter is in functional combination therewith, e.g., under transcriptional initiation regulation of the promoter.

As used herein, the “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)2, Fab, Fab′, and F(ab′)2, F(ab1)2, Fv, dAb, and Fd fragments, diabodies, and antibody-related polypeptides, so long as they exhibit the desired biological activity or function.

The term “host cell” as used herein refers to a cell into which a recombinant nucleotide sequence has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, hydrophobicity is the physical property of a substance that repels or is minimally soluble or insoluble in water. As also used herein, hydrophilicity is the physical property of a molecule that attracts water or is partially or completely soluble in water. The hydrophobicity index is a value that indicates the relative hydrophobicity or hydrophilicity of amino acid residues.

In embodiments, the hydrophobicity index is measured by the Grand Average of Hydropathy or GRAVY. This index is calculated by adding the hydrophobicity value for each residue and dividing by the length of the sequence according to the scale defined in (Kyte, J., & Doolittle, R. F. (1982), A simple method for displaying the hydropathic character of a protein; Journal of molecular biology, 157(1), 105-132). Proteins or peptides with negative GRAVY values are hydrophilic and positive values mean they are hydrophobic.

Protein variants may contain one or more conservative substitutions and/or one or more tolerated substitutions. Tolerated substitutions can be substitutions that do not fall under the definition of conservative as provided below but are nonetheless phenotypically silent. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide.

The amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulfur containing side chains). It should be appreciated that amino acid substitutions within the scope of the present disclosure can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of the nature described in the immediately preceding paragraph are often referred to as “conservative” or “semi-conservative” amino acid substitutions. The present disclosure therefore extends to use of a polypeptide comprising any of the amino acid sequences described above but with one or more conservative substitutions and or one or more tolerated substitutions in the sequence, such that the amino acid sequence of the peptide has at least 90% identity, such as 90%, 91%, 92%, 93%, 94%, 95%, 96%, 97%, 98%, 99% or 100% identity, to the peptide sequences disclosed herein.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case can be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present disclosure.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid polypeptides. BLAST protein searches can be performed with the XBLAST program, score=50, wordlength=3 to obtain amino acid sequences homologous to protein polypeptides for use in the disclosure. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between polypeptides (Id.). When utilizing BLAST, Gapped BLAST, and PSI-Blast programs, the default parameters of the respective programs (e.g., XBLAST and NBLAST) can be used. See www.ncbi.nlm.nih.gov. Another example of a mathematical algorithm utilized for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

Mutations, including conservation and tolerated substitutions, insertions, and deletions, can be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1): 30-6. The truncated casein polypeptide (TCP) sequences provided by the disclosure can be obtained from solid state synthesis, or any other appropriate method known in the art.

### INTRODUCTION

Individuals suffering from some retinal dystrophies were found to have lower levels of RdCVF protein in their eyes than did individuals without retinal dystrophies (see, e.g., PCT Publication WO02/081513). Different forms of RdCVF protein promote cone photoreceptor cell survival in vitro and in vivo. For example, intraocular injections of the short form of human RdCVF1 (RdCVF1S) protein not only rescued cone cells from degeneration but also preserved their function in animal models of inherited retinal degeneration (Yang et al. (2009) Mol Therapy 17:787-795).

Despite the promise of RdCVF protein for treatment of a variety of diseases, including ocular diseases, expression of significant levels of RdCVF at large scale and from gene therapy vectors has been challenging, e.g., see U.S. Patent Publication No. 20110034546, paragraph [0004]. Indeed, the hydrophobic nature of the protein has hampered its scalable production and purification using standard method. See, e.g., Sahel J A, Novel Treatments for Vision Disorders. Research EU Results Magazine. September 2014 (cordis.europa.eu/project/id/241683/reporting).

RdCVF has a great potential to treat eye disease including inherited retina diseases such as retinitis pigmentosa (RP) and geographic atrophy (GA). Unfortunately, the scientists in the field have encountered considerable difficulties to effectively express and secrete RdCVF protein, especially the short form RdCVF (RdCVFS) (See, e.g., Sahel J A, Novel Treatments for Vision Disorders. Research EU Results Magazine. September 2014 (cordis.europa.eu/project/id/241683/reporting).

The inventors surprisingly discovered that a fusion protein comprising a hydrophilic peptide, which in embodiments is an immunoglobulin constant region fused to a human short RdCVF and signal peptide showed marked expression and secretion compared to prior constructs known to the inventors.

### Rod-Derived Cone Viability Factor Fusion Protein

In embodiments, this invention provides a fusion protein comprising a first N-terminal signal peptide, a second peptide C-terminal to the signal peptide, and a third peptide C-terminal to the second peptide, where the second or third peptide is either an RdCVF-short peptide and the other is the constant region of an immunoglobulin. In embodiments, the second peptide is an RdCVF-short peptide, and the third peptide sequence is an immunoglobulin constant region. In embodiments, the second peptide is an immunoglobulin constant region, and the third peptide is an RdCVF-short peptide.

In embodiments, the RdCVF-short peptide is a human RdCVF-short peptide.

In embodiments, the RdCVF-short peptide is an RdCVF1-short peptide or an RdCVF2-short peptide.

In embodiments, the RdCVF-short peptide is a RdCVF1-short peptide.

In embodiments, the RdCVF1-short peptide comprises an amino acid sequence with at least 95%, 96%, 97%, 98%, 99% or 100% sequence identity to SEQ ID NO: 6.

In embodiments, the RdCVF1-short peptide comprises amino acids with at least 95%, 96%, 97%, 98%, 99% or 100% identity to amino acids 24-131 of SEQ ID NO: 4.

In embodiments, the first peptide sequence is covalently bonded to the second peptide sequence by a single peptide bond. In embodiments, the fusion protein comprises a spacer between the first peptide sequence and the second peptide sequence. In embodiments, the spacer between the first and second peptide sequences comprises from two to fourteen amino acids. In embodiments, the second peptide sequence is covalently bonded to the third peptide sequence by a single peptide bond. In embodiments, the fusion protein comprises a spacer between the second peptide sequence and the third peptide sequence. In embodiments, the spacer between the second and third peptide sequences comprises from two to four amino acids. In some embodiments, the spacer between the second and third peptide sequences comprises from two to four amino acids. In some embodiments, the fusion protein further comprises a polyadenylation signal C-terminal to the third peptide sequence.

In embodiments, the first peptide sequence is a human Igk sequence, the second peptide sequence is an RdCVF1-short sequence, and the third peptide sequence is a human IgG4-Fc sequence.

In embodiments, the fusion protein comprises an amino acid sequence with at least 95%, 96%, 97%, 98%, 99% or 100% sequence identity to SEQ ID NO: 4. In embodiments, the signal peptide, the RdCVF-short peptide, and/or the antibody peptide is not a wild-type sequence. In embodiments, the signal peptide, the RdCVF-short peptide, and the antibody peptide differs from a corresponding wild-type sequence by one or more conservative amino acid substitutions.

In some embodiments, an RdCVF protein is a fragment or an analog of an RdCVF protein that retains a cone cell and/or a rod cell survival activity or protective effect. Methods for measuring these activities or effects are known in the art. For example, (Léveillard, 2004) describes related mouse models and in vitro methods for detecting RdCVF activity. An RdCVF protein or an RdCVF coded for by a nucleic acid, can have an amino acid sequence other than a naturally-occurring amino acid sequence. For example, an RdCVF protein that is not naturally-occurring may contain amino acids in addition to those found in a naturally occurring RdCVF protein (e.g., at the amino or carboxy terminus) and/or may contain single or multiple amino acid substitutions (e.g., conservative or non-conservative amino acid substitutions) as compared to a naturally-occurring RdCVF amino acid sequence. A conservative amino acid substitution generally should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991). Conservative substitutions include, but are not limited to, those from the following groupings: Acidic Residues Asp (D) and Glu (E); Basic Residues Lys (K), Arg (R), and His (H); Hydrophilic Uncharged Residues Ser (S), Thr (T), Asn (N), and Gln (Q); Aliphatic Uncharged Residues Gly (G), Ala (A), Val (V), Leu (L), and Ile (I); Non-polar Uncharged Residues Cys (C), Met (M), and Pro (P); Aromatic Residues Phe (F), Tyr (Y), and Trp (W); Alcohol group-containing residues S and T; Aliphatic residues I, L, V and M; Cycloalkenyl-associated residues F, H, W and Y; Hydrophobic residues A, C, F, G, H, I, L, M, R, T, V, W and Y; Negatively charged residues D and E; Polar residues C, D, E, H, K, N, Q, R, S and T; Positively charged residues H, K and R; Small residues A, C, D, G, N, P, S, T and V; Very small residues A, G and S; Residues involved in turn formation A, C, D, E, G, H, K, N, Q, R, S, P and T; and Flexible residues Q, T, K, S, G, P, D, E and R.

In embodiments, the invention provides a fusion protein comprising a human short RdCVF and a human IgG4-Fc. In embodiments, the fusion protein further comprises a human Igk secretory signal sequence at its N-terminus (SEQ ID NO: 4).

There are tools publicly available to calculate a protein's hydrophobicity. In this invention, the inventors used Protein Hydrophobicity Index Analysis Program that is available on web.expasy.org/protparam to calculate hydrophobicity index for the short form human RdCVF with the human Igk signal peptide (hIgk.hRdCVF1S, SEQ ID NO: 2), and the fusion protein hIgk.hRdCVF1S.hIgG4-Fc (SEQ ID NO: 4).

In embodiments, the invention provides a fusion protein comprising a human short RdCVF and a human IgG4-Fc. In embodiments, the fusion protein further comprises a human Igk secretory signal sequence at its N-terminus (SEQ ID NO: 4).

In embodiments, the GRAVY value for hIgk.hRdCVF1S is +0.014 which is highly hydrophobic. In a great contrast, the GRAVY value for hIgk.hRdCVF1S.hIgG4-Fc is −0.353 which is highly hydrophilic. Addition of human IgG4-Fc dramatically reduced the hydrophobicity of RdCVF1S by more than 300%. In embodiments, an RdCVF-short and a hydrophilic peptide fusion protein comprising a constant region of an immunoglobulin of the invention comprises a GRAVY value of −0.005 to −2.000, −0010 to −1.500, −0.20 to −1.000, −0.30 to −0.090, −0.40 to −0.070, −0.050 to −0.600, or −0.500 to −0.580.

In embodiments, the fusion protein comprises fragments or variants of an immunoglobulin constant region. In embodiments, the fusion protein comprises 50-500 amino acids, 100-300 amino acids, 150-275 amino acids, or 220-240 amino acids of an immunoglobulin constant region, such as the constant region of IgG4. In embodiments, the fusion protein comprises a fragment of an immunoglobulin constant region that comprises less than 500, 300, or 275 amino acids. In embodiments the fusion protein comprises 220, 221, 222, 223, 224, 225, 226, 227, 228, 229, 230, 231, 232, 233, 234, 235, 236, 237, 238, 239, or 240 amino acids of an immunoglobulin constant region, such as the IgG4 constant region.

In embodiments, the fusion protein of the invention has a hydrophobicity index of less than about −0.20, −0.30, −0.40, or −0.50.

In embodiments, the fusion protein has a hydrophobicity index of −0.20 to −0.50, −0.32 to −0.36, or −0.30 to −0.40. In embodiments, the fusion protein of the invention has a hydrophobicity index of −0.20, −0.21, −0.22, −0.23, −0.24, −0.25, −0.26, −0.27, −0.28, −0.29, −0.30, −0.31, −0.32, −0.33, −0.34, −0.35, −0.36, −0.37, −0.38, −0.39, −0.40, −0.41, −0.42, −0.43, −0.44, −0.45, −0.46, −0.47, −0.48, −0.49, −0.50, −0.51, −0.52, −0.53, −0.54, −0.55, −0.56, −0.57, −0.58, −0.59, −0.60, −0.61, −0.62, −0.63, −0.64, −0.65, −0.66, −0.67, −0.68, −0.69, or −0.70.

### Rod-Derived Cone Viability Factor Fusion Protein Comprising a Secretory Signal Peptide and a Hydrophilic Peptide

In embodiments, the invention provides a fusion protein comprising a first N-terminal signal peptide, a second peptide C-terminal to the signal peptide, and a third peptide C-terminal to the second peptide, where the first peptide is a human immunoglobulin kappa chain (Igk) signal sequence, the second or third peptide is either an RdCVF-short peptide and the other is a hydrophilic peptide. In embodiments, the second peptide is an RdCVF-short peptide, and the third peptide sequence is a hydrophilic peptide. In embodiments, the second peptide is a hydrophilic peptide, and the third peptide is an RdCVF-short peptide.

In embodiments, the human Igk signal peptide comprises an amino acid sequence with at least 95%, 96%, 97%, 98%, 99% or 100% sequence identity to SEQ ID NO: 5. In embodiments, the human Igk signal sequences comprises an amino acid sequence with at least 95%, 96%, 97%, 98%, 99% or 100% sequence identity to SEQ ID NO: 8. In embodiments, the human Igk signal sequences comprises an amino acid sequence with at least 95%, 96%, 97%, 98%, 99% or 100% sequence identity to SEQ ID NO: 9.

In embodiments, the human Igk signal sequences comprises an amino acid sequence with at least 95%, 96%, 97%, 98%, 99% or 100% sequence identity to amino acids 1-23 of SEQ ID NO: 2. In embodiments, the human Igk signal sequences comprises an amino acid sequence with at least 95%, 96%, 97%, 98%, 99% or 100% sequence identity to amino acids 1-23 of SEQ ID NO: 10. In embodiments, the human Igk signal sequences comprises an amino acid sequence with at least 95%, 96%, 97%, 98%, 99% or 100% sequence identity to amino acids 1-22 of SEQ ID NO: 11.

In embodiments, the RdCVF-short peptide is a human RdCVF-short peptide. In embodiments, the RdCVF-short peptide is an RdCVF1-short peptide or an RdCVF2-short peptide. In embodiments, the RdCVF-short peptide is a RdCVF1-short peptide.

In embodiments, the RdCVF1-short peptide comprises an amino acid sequence with at least 95%, 96%, 97%, 98%, 99% or 100% sequence identity to SEQ ID NO: 6.

In embodiments, the RdCVF1-short peptide comprises amino acids with at least 95%, 96%, 97%, 98%, 99% or 100% identity to amino acids 24-131 of SEQ ID NO: 2. In embodiments, the RdCVF1-short peptide comprises amino acids with at least 95%, 96%, 97%, 98%, 99% or 100% identity to amino acids 24-131 of SEQ ID NO: 10. In embodiments, the RdCVF1-short peptide comprises amino acids with at least 95%, 96%, 97%, 98%, 99% or 100% identity to amino acids 23-131 of SEQ ID NO: 11.

In embodiments, the hydrophilic peptide is a hydrophilic protein. In embodiments, the hydrophilic peptide is a hydrophilic protein domain. In embodiments, the hydrophilic peptide is a hydrophilic oligopeptide. In embodiments, the hydrophilic peptide is a hydrophilic polypeptide. In embodiments, the hydrophilic peptide is an immunoglobulin. In embodiments, the hydrophilic peptide is a constant region of an immunoglobulin.

In embodiments, the hydrophilic peptide is a human serum albumin. In embodiments, the hydrophilic peptide comprises 200-584 amino acids, 300-580 amino acids, 400-500 amino acids, or 500-580 amino acids of HSA. In embodiments, the hydrophilic peptide comprises less than 580, 575, 565, or 500 amino acids of HSA. In embodiments the hydrophilic peptide comprises 560, 561, 562, 563, 564, 564, 566, 567, 568, 569, 570, 571, 572, 573, 574, 575, 576, 577, 578, 579, 580, 581, 582, 583, 584, 585 amino acids of HSA.

In embodiments, the hydrophilic peptide is an immunoglobulin. In embodiments, the immunoglobulin is Immunoglobulin G (IgG). In embodiments, the immunoglobulin is Immunoglobulin M (IgM). In embodiments, the immunoglobulin is Immunoglobulin A (IgA). In embodiments, the immunoglobulin is Immunoglobulin D (IgD). In embodiments, the immunoglobulin is Immunoglobulin E (IgE). In embodiments, the Immunoglobulin is a combination of IgG, IgM, IgA, IgD, and IgE. In embodiments, the Immunoglobulin is a human Immunoglobulin. In embodiments, the Immunoglobulin is not immunogenic to humans.

In embodiments, the hydrophilic peptide is an IgG1. In embodiments, the hydrophilic peptide is an IgG2. In embodiments, the hydrophilic peptide is an IgG3. In embodiments, the hydrophilic peptide is an IgG4.

In embodiments, the hydrophilic peptide is a constant region of an IgG. In embodiments, the hydrophilic peptide is a constant region of an IgM. In embodiments, the hydrophilic peptide is a constant region of an IgA. In embodiments, the hydrophilic peptide is a constant region of an IgD. In embodiments, the hydrophilic peptide is a constant region of an IgE.

In embodiments, the hydrophilic peptide is a constant region of an IgG1. In embodiments, the hydrophilic peptide is a constant region of an IgG2. In embodiments, the hydrophilic peptide is a constant region of an IgG3. In embodiments, the hydrophilic peptide is a constant region of an IgG4.

In embodiments, the fusion protein comprises fragments or variants of an immunoglobulin constant region. In embodiments, the fusion protein comprises 50-500 amino acids, 100-300 amino acids, 150-275 amino acids, or 220-240 amino acids of an immunoglobulin constant region, such as the constant region of IgG4. In embodiments, the fusion protein comprises a fragment of an immunoglobulin constant region that comprises less than 500, 300, or 275 amino acids. In embodiments the peptide fusion protein comprises 220, 221, 222, 223, 224, 225, 226, 227, 228, 229, 230, 231, 232, 233, 234, 235, 236, 237, 238, 239, or 240 amino acids of an immunoglobulin constant region, such as the IgG4 constant region.

In embodiments, the immunoglobulin is a human IgG4, comprising an amino acid sequence with at least 95%, 96%, 97%, 98%, 99% or 100% sequence identity to SEQ ID NO: 7. In some embodiments the IgG4 comprises amino acids with at least 95%, 96%, 97%, 98%, 99% or 100% identity to amino acids 132-357 of SEQ ID NO: 4.

In embodiments, the first peptide sequence is a human Igk sequence, the second peptide sequence is an RdCVF1-short sequence, and the third peptide sequence is a hydrophilic peptide. In embodiments, the hydrophobicity index of the hydrophilic peptide is less than that of a human serum albumin peptide (HSA). In embodiments, the hydrophobicity index of the hydrophilic peptide is less than about −0.20, −0.30, −0.40, −0.50, −0.60 or −0.70. In embodiments, the hydrophilic peptide has a hydrophobicity index of −0.20 to −0.70, −0.54 to −0.58, or −0.5 to −0.60. In embodiments, the hydrophilic peptide of the invention has a hydrophobicity index of −0.20, −0.21, −0.22, −0.23, −0.24, −0.25, −0.26, −0.27, −0.28, −0.29, −0.30, −0.31, −0.32, −0.33, −0.34, −0.35, −0.36, −0.37, −0.38, −0.39, −0.40, −0.41, −0.42, −0.43, −0.44, −0.45, −0.46, −0.47, −0.48, −0.49, −0.50, −0.51, −0.52, −0.53, −0.54, −0.55, −0.56, −0.57, −0.58, −0.59, −0.60, −0.61, −0.62, −0.63, −0.64, −0.65, −0.66, −0.67, −0.68, −0.69, or −0.70.

In embodiments, the hydrophilic peptide comprises 50-500 amino acids, 100-300 amino acids, 150-275 amino acids, or 220-240 amino acids. In embodiments, the hydrophilic peptide comprises less than 500, 300, or 275 amino acids. In embodiments the hydrophilic peptide comprises 220, 221, 222, 223, 224, 225, 226, 227, 228, 229, 230, 231, 232, 233, 234, 235, 236, 237, 238, 239, or 240 amino acids.

In embodiments, the hydrophilic peptide of this invention is a constant region of an Immunoglobulin G (IgG-Fc). In embodiments, the IgG-Fc has a hydrophobicity index of −0.20 to −0.70, −0.30 to −0.60, or −0.40 to −0.57. In embodiments, the IgG4-Fc of the invention has a hydrophobicity index of −0.20, −0.21, −0.22, −0.23, −0.24, −0.25, −0.26, −0.27, −0.28, −0.29, −0.30, −0.31, −0.32, −0.33, −0.34, −0.35, −0.36, −0.37, −0.38, −0.39, −0.40, −0.41, −0.42, −0.43, −0.44, −0.45, −0.46, −0.47, −0.48, −0.49, −0.50, −0.51, −0.52, −0.53, −0.54, −0.55, −0.56, −0.57, −0.58, −0.59, −0.60, −0.61, −0.62, −0.63, −0.64, −0.65, −0.66, −0.67, −0.68, −0.69, or −0.70. In embodiments, the IgG constant region has a hydrophobicity index of −0.568.

In embodiments, an RdCVF-short and a hydrophilic peptide fusion protein comprising an Igk signal sequence comprises a GRAVY value of −0.005 to −2.000, −0010 to −1.500, −0.20 to −1.000, −0.30 to −0.090, −0.40 to −0.070, −0.050 to −0.600, or −0.500 to −0.580.

In embodiments, the first peptide sequence is covalently bonded to the second peptide sequence by a single peptide bond. In embodiments, the fusion protein comprises a spacer between the first peptide sequence and the second peptide sequence. In embodiments, the spacer between the first and second peptide sequences comprises from two to fourteen amino acids. In embodiments, the second peptide sequence is covalently bonded to the third peptide sequence by a single peptide bond. In embodiments, the fusion protein comprises a spacer between the second peptide sequence and the third peptide sequence. In embodiments, the spacer between the second and third peptide sequences comprises from two to four amino acids. In some embodiments, the spacer between the second and third peptide sequences comprises from two to four amino acids. In some embodiments, the fusion protein further comprises a polyadenylation signal C-terminal to the third peptide sequence.

In embodiments, the first peptide sequence is a human Igk sequence, the second peptide sequence is an RdCVF1-short sequence, and the third peptide sequence is the constant region of IgG4 (IgG4-Fc).

In embodiments, the fusion protein comprises an amino acid sequence with at least 95%, 96%, 97%, 98%, 99% or 100% sequence identity to SEQ ID NO: 4. In embodiments, the fusion protein comprises an amino acid sequence with at least 95%, 96%, 97%, 98%, 99% or 100% sequence identity to SEQ ID NO: 12. In embodiments, the fusion protein comprises an amino acid sequence with at least 95%, 96%, 97%, 98%, 99% or 100% sequence identity to SEQ ID NO: 13. In embodiments, the Igk peptide, the RdCVF-short peptide, and/or the antibody peptide is not a wild-type sequence. In embodiments, the signal peptide, the RdCVF-short peptide, and the antibody peptide differs from a corresponding wild-type sequence by one or more conservative amino acid substitutions.

### Recoded RdCVF Coding Sequences

In embodiments, the present disclosure provides a fusion protein comprising human Igk signal sequence, an RdCVF protein peptide, and an immunoglobulin constant region peptide, the human Igk signal peptide comprises an amino acid sequence with at least 95% sequence identity to SEQ ID NO: 4, wherein the RdCVF coding sequence comprises a recoded nucleotide sequence. In embodiments, the RdCVF recoded nucleotide sequence lacks an initiating methionine.

The term “recoded” or “recoded nucleotide sequence” means that at least one native codon is changed to a different codon that encodes for the same amino acid as the native codon. In some embodiments, a recoded RdCVF coding region has at least 2.5%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least or at least 95% of the codons recoded. In some embodiments, about 20-50%, 35-45%, 38-42% or 39-41% or the codons are recoded. In some embodiments, a recoded codon is replaced with a codon that is more prevalently used in humans. In some embodiments, at least 5%, at least 10%, at least 15%, at least 20%, at least 25%, at least 30%, at least 35%, at least 40%, at least 45%, at least 50% or at least 55% of the codons have been replaced with a codon that is more prevalently used in humans.

In some embodiments, a recoded sequence has between about 70-90%, about 75-85%, about 80-85% or about 82-85% identity with the corresponding native coding sequence. In some embodiments, a recoded nucleotide sequence has at least 15% of the nucleotides different as compared to a corresponding native nucleotide sequence. In some embodiments, a recoded nucleotide sequence is less than 90% identical to a corresponding native nucleotide sequence.

Recoding can also be used to change the chemical make-up of a DNA and/or an RNA coding sequence such as the guanine/cytosine (GC) percentage. In some embodiments, recoding of an RdCVF coding region raises the GC content to at least 60%. In some embodiments, a recoded RdCVF coding region has a GC percentage between 60-64% or 60.4%-63.5%. Recoding can be used to change the secondary structure of mRNA. Recoding can also be used to remove or add particular motifs or sites to a coding sequence or nucleic acid molecule, such as procarya inhibitory motifs, consensus splice donor sites, cryptic splice donor sites or a combination thereof. In some embodiments, a recoded RdCVF coding sequence has less procarya inhibitory motifs, consensus splice donor sites, cryptic splice donor sites or a combination thereof than the native sequence. In some embodiments, a recoded RdCVF coding sequence contains no procarya inhibitory motifs, no consensus splice donor sites and/or no cryptic splice donor sites.

In some embodiments of the invention, a recoded RdCVF coding sequence does not contain the initial RdCVF ATG codon and/or RdCVF stop codon (e.g., TAG). For example, an RdCVF recoded coding sequence can be operatively linked 5′ or 3′ to another coding sequence resulting in a protein comprising a heterologous amino acid sequence, N-terminal and/or C-terminal to the RdCVF amino acid sequence, respectively. In some of these embodiments, the initial RdCVF ATG codon and/or RdCVF stop codon may be deleted or present in the RdCVF coding region. If another coding sequence is fused in frame at the 3′end of an RdCVF coding region, then the native RdCVF stop codon will not typically be present at the end of the RdCVF coding sequence.

In some embodiments, a recoded RdCVF coding sequence is a recoded sequence that codes for amino acids 24-234 of SEQ ID NO: 3.

### Signal Peptides/Secretion Signals

The protein secretory pathway is utilized by more than a quarter of the human proteome. Signal peptide plays an essential role in guiding a newly synthesized protein to translocate from cytosol through the secretory pathway to a different location, e.g., cell membrane for a transmembrane protein or outside of the cells for a secreted protein. Signal peptides are highly diverse. In humans alone, more than 3000 proteins have been identified so far that contain different signal peptides. The highly diverse primary sequences of these signal peptides suggest that they play a role in regulating the physiological level of secretion of a particular protein from the cell (Kober, L. 2013).

Simply adjoining a heterologous signal peptide to a mature protein sequence does not guarantee secreted expression, and expression of a protein when placed directly adjacent to a non-native signal peptide could be completely abrogated (Guler-Cane, 2016). To add another layer of complexity, the efficiency of a signal peptide in guiding a protein secretion is also affected by the amino acid of the mature protein.

Endogenous human RdCVF does not have an identifiable signal peptide. A mouse Igk signal peptide was demonstrated to mediate efficient secretion of RdCVF-S (U.S. Pat. No. 9,265,813). The mouse Igk signal sequence was used to reduce potential immunogenicity of a human signal sequence in in vivo studies in mice. Since there is no significant homology between mouse and human Igk signal peptide, the disclosure provides human Igk signal peptides that are capable of mediating expression and secretion of RdCVF-S. Utilization of a signal peptide of human origin is expected to avoid immunogenicity associated with a mouse Igk signal peptide when the gene therapy vector is tested in human studies.

Signal sequences are translated in frame as a peptide attached, typically, to the amino-terminal end of a polypeptide of choice. A secretory signal sequence will cause the secretion of the polypeptide from the cell by interacting with the machinery of the host cell. As part of the secretory process, this secretory signal sequence will typically be cleaved off or at least partially cleaved off. The term “signal peptide” can also refer to a signal sequence or a nucleic acid sequence encoding the signal peptide.

The structure of a typical signal peptide can include three distinct regions: (i) an N-terminal region that contains a number of positively charged amino acids (e.g., lysine and arginine); (ii) a central hydrophobic core region (h-region); (iii) a hydrophilic cleavage region (c-region) that contains the sequence motif recognized by the signal peptidase. (von Heijne, 1983; von Heijne, 1985; von Heijne, 1997) These signal peptides can be used in accordance with the invention. In some embodiments, the signal peptide is from an immunoglobulin such as an Igk.

A signal sequence can be a mammalian, murine or human signal sequence. In embodiments, the signal peptide refers to a human signal peptide.

In embodiments, the signal peptide is an immunoglobulin kappa chain (Igk) signal peptide, a human growth hormone (HGH), a brain-derived neurotrophic factor (BDNF), an insulin growth factor 1 (IGF-1), a β-glucuronidase (GUSB), or an albumin signal peptide.

In embodiments, the signal peptide in the fusion protein is an Igk signal peptide.

In embodiments, the human Igk signal peptide comprises an amino acid sequence with at least 95%, 96%, 97%, 98%, 99% or 100% sequence identity to SEQ ID NO: 5. In embodiments, the human Igk signal peptide comprises amino acids with at least 95%, 96%, 97%, 98%, 99% or 100% identity to amino acids 1-23 of SEQ ID NO: 4. A nucleotide sequence coding for a signal peptide can be a wild-type sequence or it can be a recoded sequence.

In some embodiments of the invention, a signal peptide sequence is operatively linked at the N-terminal or C-terminal of an RdCVF, e.g., RdCVF1-S or RdCVF2-S. In some embodiments, the signal peptide directs transit of the protein to secretory pathways, e.g., to the endoplasmic reticulum (ER). In some embodiments, a signal peptide facilitates protein transport from the cytoplasm to destinations outside the cell. Signal peptide sequences may be selected from naturally occurring signal peptide sequences, derivatives thereof, or a synthetic designed sequence. In some embodiments, non-limiting parameters for a designed signal peptide sequences include a sequence of 3-40 residues, comprising a 3- to 20-residue hydrophobic core flanked by several relatively hydrophilic residues

The protein secretory pathway is utilized by more than a quarter of the human proteome. Signal peptide plays an essential role in guiding a newly synthesized protein to translocate from cytosol through the secretory pathway to a different location, e.g., cell membrane for a transmembrane protein or outside of the cells for a secreted protein. Signal peptides are highly diverse. In humans alone, more than 3000 proteins have been identified so far that contain different signal peptides. The highly diverse primary sequences of these signal peptides suggest that they play a role in regulating the physiological level of secretion of a particular protein from the cell (Kober, L. 2013).

Simply adjoining a heterologous signal peptide to a mature protein sequence does not guarantee secreted expression, and expression of a protein when placed directly adjacent to a non-native signal peptide could be completely abrogated (Guler-Cane, 2016). To add another layer of complexity, the efficiency of a signal peptide in guiding a protein secretion is also affected by the amino acid of the mature protein.

Endogenous human RdCVF does not have an identifiable signal peptide. A mouse Igk signal peptide was demonstrated to mediate efficient secretion of RdCVF-S (U.S. Pat. No. 9,265,813). The mouse IgK signal sequence was used to reduce potential immunogenicity of a human signal sequence in in vivo studies in mice. Since there is no significant homology between mouse and human Igk signal peptide, the disclosure provides human Igk signal peptides that are capable of mediating expression and secretion of RdCVF-S. Utilization of a signal peptide of human origin avoids immunogenicity associated with a mouse Igk signal peptide when the gene therapy vector is tested in human studies.

Signal sequences are translated in frame as a peptide attached, typically, to the amino-terminal end of a polypeptide of choice. A secretory signal sequence will cause the secretion of the polypeptide from the cell by interacting with the machinery of the host cell. As part of the secretory process, this secretory signal sequence will typically be cleaved off or at least partially cleaved off. The term “signal peptide” can also refer to a signal sequence or a nucleic acid sequence encoding the signal peptide.

The structure of a typical signal peptide can include three distinct regions: (i) an N-terminal region that contains a number of positively charged amino acids (e.g., lysine and arginine); (ii) a central hydrophobic core region (h-region); (iii) a hydrophilic cleavage region (c-region) that contains the sequence motif recognized by the signal peptidase. (von Heijne, 1983; von Heijne, 1985; von Heijne, 1997) These signal peptides can be used in accordance with the invention. In some embodiments, the signal peptide is from an immunoglobulin such as an Igk.

A signal sequence can be a mammalian, murine or human signal sequence. In embodiments, the signal peptide refers to a human signal peptide.

In embodiments, the signal peptide is an immunoglobulin kappa chain (Igk) signal peptide, a human growth hormone (HGH), a brain-derived neurotrophic factor (BDNF), an insulin growth factor 1 (IGF-1), a β-glucuronidase (GUSB), or an albumin signal peptide.

In embodiments, the signal peptide in the fusion protein is an Igk signal peptide.

In embodiments, the use of the human Igk peptide results in the expression of a polypeptide that has surprisingly superior expression and secretion.

In embodiments, the human Igk signal peptide comprises an amino acid sequence with at least 95%, 96%, 97%, 98%, 99% or 100% sequence identity to SEQ ID NO: 5. In embodiments, the human Igk signal peptide comprises amino acids with at least 95%, 96%, 97%, 98%, 99% or 100% identity to amino acids 1-23 of SEQ ID NO: 4. A nucleotide sequence coding for a signal peptide can be a wild-type sequence or it can be a recoded sequence.

In some embodiments of the invention, a signal peptide sequence is operatively linked at the N-terminal or C-terminal of an RdCVF, e.g., RdCVF1-S or RdCVF2-S. In some embodiments, the signal peptide directs transit of the protein to secretory pathways, e.g., to the endoplasmic reticulum (ER). In some embodiments, a signal peptide facilitates protein transport from the cytoplasm to destinations outside the cell. Signal peptide sequences may be selected from naturally occurring signal peptide sequences, derivatives thereof, or a synthetic designed sequence. In some embodiments, non-limiting parameters for a designed signal peptide sequences include a sequence of 3-40 residues, comprising a 3- to 20-residue hydrophobic core flanked by several relatively hydrophilic residues

### Immunoglobulins/the Constant Region of Immunoglobulins

In embodiments, the fusion protein, nucleic acid, or expression vector of this invention comprises a constant region of an immunoglobulin. The constant region of an immunoglobulin (Ig), also known as the Fc (fragment crystallizable) region, is the portion of the antibody that does not vary among different antibodies of the same class or subclass. It is responsible for mediating many of the effector functions of antibodies, such as binding to receptors on immune cells (Fc receptors) and interacting with complement proteins, which are essential for the immune response. The constant region is located at the carboxyl-terminal end of both the heavy and light chains of the immunoglobulin, but its structure is most crucial on the heavy chains.

In embodiments, the immunoglobulin constant region in the fusion proteins of the invention is Immunoglobulin G (IgG), Immunoglobulin M (IgM), Immunoglobulin A (IgA), Immunoglobulin D (IgD), Immunoglobulin E (IgE), or a combination thereof. In embodiments, the immunoglobulin is a human immunoglobulin. In embodiments, the immunoglobulin is not immunogenic to humans.

In embodiments, the IgG is an IgG1, IgG2, IgG3, IgG4, or a combination thereof.

In embodiments, the immunoglobulin is a human IgG4, comprising an amino acid sequence with at least 95%, 96%, 97%, 98%, 99% or 100% sequence identity to SEQ ID NO: 7. In some embodiments the IgG4 comprises amino acids with at least 95%, 96%, 97%, 98%, 99% or 100% identity to amino acids 132-357 of SEQ ID NO: 4.

### Nucleic Acid Encoding an RdCVF Fusion Protein

The invention includes nucleic acids comprising a nucleotide sequence encoding an RdCVF and includes vectors comprising these nucleic acids.

To ensure local and/or long-term expression of a nucleic acid of interest, some embodiments of the invention contemplate transducing a cell with a nucleic acid or vector encoding an RdCVF. The instant invention is not to be construed as limited to any one particular nucleic delivery method, and any available nucleic acid delivery vehicle with either an in vivo or in vitro nucleic acid delivery strategy, or the use of manipulated cells (such as the technology of Neurotech, Lincoln, R I, e.g., see U.S. Pat. Nos. 6,231,879; 6,262,034; 6,264,941; 6,303,136; 6,322,804; 6,436,427; 6,878,544) as well as nucleic acids of the invention encoding an RdCVF per se (e.g., “naked DNA”), can be used in the practice of the invention. Various delivery vehicles, such as vectors, can be used with the invention. For example, viral vectors, amphitrophic lipids, cationic polymers, such as polyethylenimine (PEI) and polylysine, dendrimers, such as combburst molecules and starburst molecules, nonionic lipids, anionic lipids, vesicles, liposomes and other synthetic nucleic acid means of delivery (e.g., see U.S. Pat. Nos. 6,958,325 and 7,098,030; Langer, 1990; Lopez-Berestein, 1989; Wang, 2023) “naked” nucleic acids and so on can be used in the practice of the instant invention.

In some embodiments, a nucleic acid molecule is used in which the RdCVF coding sequences and any other desired sequences are flanked by regions that promote homologous recombination at a desired site in the genome, thus providing for intrachromosomal expression of the RdCVF nucleic acid (Koller et al., (1989) Proc. Natl. Acad. Sci. USA 86:8932-8935; Zijlstra et al. (1989) Nature 342:435¬438). Delivery of a nucleic acid into a subject may be either direct, in which case the subject is directly exposed to the nucleic acid or nucleic acid-carrying vector, or indirect, in which case, cells are first transformed with the nucleic acid in vitro, then transplanted into the subject.

A vector is a means by which a nucleic acid of interest (e.g., a therapeutic nucleic acid that can encode a therapeutic protein) is introduced into a target cell of interest. Methods for obtaining or constructing a vector of interest include, but are not limited to, standard gene manipulation techniques, sequencing reactions, restriction enzymes digests, polymerase reactions, PCR, PCR SOEing, ligations, recombinase reactions (e.g., Invitrogen's GATEWAY® technology) other enzymes active on nucleic acids, bacteria and virus propagation materials and methods, chemicals and reagents, site directed mutagenesis protocols and so on, as known in the art, see, for example, the Maniatis et al. text, “Molecular Cloning.”.

Nucleic acids of the invention will typically comprise a promoter sequence operatively linked to human Igk signal sequence and human RdCVF coding sequence. A promoter may be a tissue specific promoter, a cell specific promoter, an inducible promoter, a repressible promoter, a constitutive promoter, a synthetic promoter or a hybrid promoter, for example. Examples of promoters useful in the constructs of the invention include, but are not limited to, a phage lambda (PL) promoter; an SV40 early promoter; a herpes simplex viral (HSV) promoter; a cytomegalovirus (CMV) promoter, such as the human CMV immediate early promoter; a hybrid promoter with CMV enhancer and chicken beta-actin promoter; a tetracycline-controlled trans-activator-responsive promoter (tet) system; a long terminal repeat (LTR) promoter, such as a MoMLV LTR, BIV LTR or an HIV LTR; a U3 region promoter of Moloney murine sarcoma virus; a Granzyme A promoter; a regulatory sequence(s) of the metallothionein gene; a CD34 promoter; a CD8 promoter; a thymidine kinase (TK) promoter; a B19 parvovirus promoter; a PGK promoter; a glucocorticoid promoter; a heat shock protein (HSP) promoter, such as HSP65 and HSP70 promoters; an immunoglobulin promoter; an MMTV promoter; a Rous sarcoma virus (RSV) promoter; a lac promoter; a CaMV 35S promoter; and a nopaline synthetase promoter. In some embodiments, a promoter is an MND promoter (Robbins, 1997), or an MNC promoter, which is a derivative of the MND promoter in which the LTR enhancers are combined with a minimal CMV promoter (Haberman, 2000).

In some embodiments, a vector or nucleic acid of the invention comprises an intron, operatively linked to a coding sequence for an RdCVF protein. An intron can be from an RdCVF gene or be a heterologous intron. Heterologous introns are known and non-limiting examples include a human β-globin gene intron and a beta-actin intron. In some embodiments, an intron sequence is a human β-globin gene intron sequence.

In some embodiments, a nucleic acid of the invention comprises a nucleotide sequence encoding a coding sequence for an RdCVF protein, wherein the RdCVF coding sequence comprises a recoded nucleotide sequence. A nucleic acid can encode for an RdCVF1 protein and/or an RdCVF2 protein. In embodiments, the RdCVF protein is an RdCVF1-Short (RdCVF1S) or RdCVF2-Short (RdCVF2-S) protein. In some embodiments, the RdCVF protein is a human RdCVF1-S, or RdCVF2-S protein,

Typically a mammalian nucleotide coding region starts with the nucleotide sequence ATG (initiating methionine codon), such as found in a human RdCVF coding region. As discussed herein, some embodiments of the invention provide a recoded RdCVF coding region and in some further embodiments the coding region is fused, in-frame with a second coding region, e.g., a coding sequence for a signal sequence. In some of these cases, the ATG nucleotide sequence is not necessarily at the start of the RdCVF coding region, e.g., the RdCVF coding region starts by coding for the second amino acid of the particular RdCVF protein. However, the ATG nucleotide sequence can be at the start of the RdCVF coding region, even when the RdCVF coding region is operatively linked to another coding region 5′ to the RdCVF coding region.

In embodiments, the present disclosure provides a nucleic acid comprising a nucleotide sequence encoding a fusion protein comprising a first N-terminal signal peptide, a second peptide C-terminal to the signal peptide, and a third peptide C-terminal to the second peptide sequence. In embodiments, the second peptide or the third peptide sequence is an RdCVF-short peptide sequence and the other is an immunoglobulin constant region sequence. In embodiments, the second peptide is an RdCVF-short peptide, and the third peptide is an immunoglobulin constant region. In embodiments, the second peptide is an immunoglobulin constant region, and the third peptide sequence is an RdCVF-short peptide. In some embodiments, a nucleic acid of the invention encodes a fusion protein comprising SEQ ID NO: 4.

In embodiments, the nucleic acid comprises a first peptide sequence that is covalently bonded to the second peptide sequence by a single peptide bond. In embodiments, the nucleic acid comprises a spacer between the first peptide sequence and the second peptide sequence. In embodiments, the spacer between the first and second peptide sequences comprises from two to fourteen amino acids. In embodiments, the second peptide sequence is covalently bonded to the third peptide sequence by a single peptide bond. In embodiments, the nucleic acid comprises a spacer between the second peptide sequence and the third peptide sequence. In embodiments, the spacer between the second and third peptide sequences comprises from two to four amino acids. In some embodiments, the spacer between the second and third peptide sequences comprises from two to four amino acids. In some embodiments, the nucleic acid further comprises a polyadenylation signal C-terminal to the third peptide sequence.

In embodiments, the nucleic acid encodes a first peptide sequence which is a human Igk sequence, a second peptide sequence which is an RdCVF1-short sequence, and a third peptide sequence which is a human IgG4-Fc sequence.

In embodiments, the nucleic acid comprises an amino acid sequence with at least 95%, 96%, 97%, 98%, 99% or 100% sequence identity to SEQ ID NO: 4. In embodiments, the signal peptide, the RdCVF-short peptide, and/or the antibody peptide is not a wild-type sequence. In embodiments, the signal peptide, the RdCVF-short peptide, and the antibody peptide differ from a corresponding wild-type sequence by one or more conservative amino acid substitutions.

In some embodiments a nucleic acid of the invention comprises a coding region for an RdCVF, wherein the RdCVF coding sequence has been recoded.

In some embodiments of the invention, a nucleic acid of the invention is in a vector, such as a viral vector.

### Viral Vectors

The invention includes viral vectors comprising the nucleic acid of the invention, e.g., a vector comprising a nucleic acid comprising a nucleotide sequence encoding an fusion protein with a human Igk signal peptide, RdCVF peptide, and an IgG4-Fc peptide, a human Igk signal sequence, wherein the nucleic acid sequence has at least 95% sequence identity to SEQ ID NO: 3. Examples of viral vectors useful in the present invention are described in PCT Publication No. WO08/106644 and U.S. Patent Publication No. US20100120665. In some embodiments, the invention is not limited to a particular viral vector. Viral vectors include, but are not limited to, retroviral vectors, lentiviral vectors, adenoviral vectors (see, for example, U.S. Pat. No. 7,045,344), AAV vectors (e.g., see U.S. Pat. No. 7,105,345), Herpes viral vectors (e.g., see U.S. Pat. Nos. 5,830,727 and 6,040,172), hepatitis (e.g., hepatitis D) viral vectors (e.g., see U.S. Pat. No. 5,225,347), SV40 vectors, EBV vectors (e.g., see U.S. Pat. No. 6,521,449) and Newcastle disease virus vectors (e.g., see U.S. Pat. Nos. 6,146,642, 7,442,379, 7,332,169 and 6,719,979). In some embodiments, a lentiviral vector is an HIV, EIAV, SIV, FIV or BIV vector. In some embodiments, a vector is selected from an AAV vector or an adenoviral vector

The invention also provides a cell that produces a viral vector of the invention. In embodiments, the cell that produces a viral vector of the invention is a 293 cell, a CHO cell, a PerC6 cell, a Vero cell, a BHK cell, a HeLa cell, a COS cell, a MDCK cell, a 3T3 cell and a W138.

Vector virions of the invention may be administered in vivo or in vitro to cells (e.g., mammalian cells). Vectors (viral or nonviral) can be used to transduce or transform cells including, but not limited to, undifferentiated cells, differentiated cells, somatic cells, primitive cells and/or stem cells.

In some embodiments, a viral vector of the invention comprises a decay accelerating factor (DAF). For example, an enveloped viral vector includes a DAF on the viral membrane. In some embodiments, a DAF is a wild-type DAF. In some embodiments, a DAF is part of a fusion protein with an envelope protein, e.g., see Guibinga et al. Mol Ther. 2005 11(4):645-51.

Adenovirus is a non-enveloped, nuclear DNA virus with a genome typically of about 36 kb. The human adenoviruses are divided into numerous serotypes (approximately 47, numbered accordingly and classified into 6 groups: A, B, C, D, E and F.

Recombinant adenoviral vectors have tropism for both dividing and non-dividing cells, minimal pathogenic potential, ability to replicate to high titer for preparation of vector stocks and the potential to carry relatively large nucleotide sequence inserts (Berkner, (1992) Curr. Top. Micro. Immunol. 158:39-66; Jolly, (1994) Cancer Gene Therapy 1:51-64). Adenoviral vectors with deletions of various adenoviral gene sequences have been designed as suitable vehicles for delivery of nucleic acids to cells. In some embodiments, an adenoviral vector of the invention is a helper dependent or a “gutless” adenoviral vector. Adenoviral vectors can be used that are deleted in one or more of the following genes: Ela, E1b, E2a, E2b and E3. Methods for conducting adenovirus-based nucleic acid delivery are described in, e.g., U.S. Pat. Nos. 5,824,544; 5,868,040; 5,871,722; 5,880,102; 5,882,877; 5,885,808; 5,932,210; 5,981,225; 5,994,106; 5,994,132; 5,994,134; and 6,001,557.

AAV vectors are derived from single-stranded (ss) DNA parvoviruses. A single AAV particle can accommodate up to 5 kb of ssDNA, leaving about 4.5 kb for a transgene and regulatory elements. Trans-splicing systems as described, for example, in U.S. Pat. No. 6,544,785, may nearly double this limit and these types of vectors may also be used with the invention. With regard to the invention, essentially AAV of any serotype can be used. In some embodiments of the invention, an AAV1, AAV2, AAV3, AAV4, AAV5, AAV6, AAV7, AAV8, AAV9 or AAVrh74 serotype may be used (e.g., see U.S. Pat. Nos. 5,173,414, 5,252,479, 5,552,311, 5,658,776, 5,658,785, 5,763,416, 5,773,289, 5,843,742, 5,869,040, 5,942,496, 5,948,675, 6,001,650 and 7,790,449; PCT Publication No. WO2009134681; Kassim et al., PLoS ONE (2010) 5(10)e13424:1-10; Kotin, Hum Mol Genet (2011) 20(R1):R2-6; Shoti et al., Mol Ther Methods Clin Dev (2023) 31:101147), although the invention is not limited to these serotypes (see, e.g., Gao et al. (2002) PNAS 99:11854-11859; and Viral Vectors for Gene Therapy: Methods and Protocols, ed. Machida, Humana Press, 2003.

An AAV vector of the invention may also be pseudotyped. Pseudotyped AAV vectors contain the genome of one AAV serotype in the capsid of a second AAV serotype (e.g., see Auricchio et al., (2001) Hum. Mol. Genet., 10(26):3075-81). An AAV vector of the invention may contain a mutated capsid and/or be retargeted. For example, see Grieger et al. (Adv Biochem Eng Biotechnol. (2005) 99:119-45); Gonçalves et al. (Mol Ther. (2006) 13(5):976-86); and Warrington et al. (J Virol. (2004) 78(12):6595-609).

In some embodiments of the invention, an AAV vector is coated with polymers, e.g., reactive polymers to reduce natural tropism or natural binding of the AAV vector; to retarget the AAV vector and/or to provide resistance to neutralizing antisera. For example, see Carlisle et al. (J Gene Med. (2008) 10(4):400-11).

Further, AAV vectors derived from different serotypes of AAV can be modified in the capsid proteins by incorporating a peptide to enhance the vector's transduction efficiency, change the tropism for tissue and organ, reduce immunogenicity, and evade neutralizing antibodies against the viral vector. AAV capsid variable regions IV and VIII are permissive to capsid modification (Havlik et al., J. Virology, 94:e00976 (2020); Havlik et al., J. Virology, 95:e0058721 (2021); Becker et al., Pathogens, 11:756 (2022); Gonzalez et al., Nat Commun., 13:5947 (2022).

Retroviruses are RNA viruses wherein the viral genome is RNA. When a host cell is infected with a retrovirus, the genomic RNA is reverse transcribed into a DNA intermediate which is integrated efficiently into the chromosomal DNA of infected cells. Lentiviruses contain other genes with regulatory or structural function.

A DNA viral vector is a viral vector based on or derived from a virus that has a DNA based genome. A non-enveloped virus viral vector is a viral vector based on or derived from a virus that lacks a lipid-bilayer membrane.

In some embodiments, a viral vector of the invention is an AAV vector. In some embodiments, a viral vector of the invention is not a bovine immunodeficiency viral vector or it is not a lentiviral vector. In some embodiments, a viral vector is selected from the group consisting of a DNA viral vector, a non-enveloped viral vector and an adenoviral vector.

Tremendous success of lipid nanoparticles (LNP) delivery technology in COVID-19 vaccine has proven LNP's utility as a non-viral vector delivery platform. LNP has also been used to deliver mRNA, siRNA, antisense oligonucleotides, microRNA and DNA in various preclinical studies (Hald Albertsen et al., Adv Drug Deliv Rev. 188:114416 (2022)). Nucleic acid sequence coding RdCVF could be delivered via LNP technology.

In some embodiments the expression vector of this invention is operatively linked to a control sequence. In some embodiments, the control sequence is a promoter. In some embodiments, the promoter is a CMV promoter.

In embodiments, the vector of this invention is a plasmid. In embodiments, the vector is an AAV expression plasmid. In embodiments, the AAV expression plasmid is an AAV8 expression plasmid.

In some embodiments, the expression vector of this invention has at least 95%, 96%, 97%, 98%, 99% or 100% sequence identity to SEQ ID NO: 3.

In embodiments, the vector is a viral vector. In some embodiments, the vector is an AAV vector, a Lentiviral vector, a retroviral vector, an Adenoviral vector, or a synthetic viral vector.

### Cellular Delivery of RdCVF, Including Encapsulated Cells

Another approach to gene therapy or protein delivery involves transferring a gene to cells in vitro or ex vivo and then administering the cells to a mammal or subject. Transferring a nucleic acid to cells can be by any method, such as, transfection, microinjection, electroporation, cell fusion, chromosome-mediated gene transfer, microcell-mediated gene transfer, spheroplast fusion, lipofection, microparticle bombardment, calcium phosphate mediated transfection, viral vector or bacteriophage transduction and so on. Optionally, a selectable marker also can be introduced into the cells. If a selectable marker is utilized, the cells can be then placed under selection, e.g., to enhance expression and/or to isolate/select those cells that express the transferred coding region (see, e.g., Loeffler & Behr, Meth. Enzymol. 217:599-618 (1993); Cohen et al., Meth. Enzymol. 217:618-644 (1993); and Cline, Pharmac. Ther. 29:69 92 (1985)). Those cells can then be delivered to a subject directly or after encapsulation.

In some embodiments, a nucleic acid is introduced into a cell prior to in vivo administration of the resulting recombinant cell. In some embodiments, a technique can provide for the stable transfer of the nucleic acid to the cell, so that the nucleic acid is expressible by the cell and in some cases heritable and expressible by its cell progeny. Recombinant cells can be delivered to a subject by various methods. In some embodiments, an RdCVF protein is expressed from a cell via a regulatable, inducible and/or repressible promoter.

In some embodiments, a cell used is autologous, allogeneic or xenogeneic with regard to a subject. In some embodiments, autologous cells are manipulated ex vivo to cause them to contain a nucleic acid of the invention which allows the cell to produce or secrete an RdCVF protein and the cells are introduced back to the subject.

In some embodiments, cells are administered locally (e.g., in a joint, intravitreal, intraretinal, intracranially etc.) or systemically (e.g., Intravenously).

In some embodiments, recombinant blood cells (e.g., hematopoietic stem and/or progenitor cells) are administered intravenously. In some embodiments, eye cells and/or pluripotential cells can be injected directly into the eye.

A stem- and/or progenitor cell which can be isolated and maintained in vitro can potentially be used in accordance with some embodiments of the invention. Such stem cells include, but are not limited, to hematopoietic stem cells (HSC), stem cells of epithelial tissues such as the skin and the lining of the gut, embryonic heart muscle cells, liver stem cells (see, e.g., WO 94/08598), and neural stem cells (e.g., Stemple and Anderson (1992) Cell 71:973-985). In some embodiments, the administered cell is a stem cell comprising a nucleic acid of the invention and is capable of expressing and secreting an RdCVF.

Encapsulated cells can allow controlled and/or continuous delivery of a protein, such as RdCVF, in vivo. In some embodiments, cells comprising a nucleic acid of the invention and expressing and/or secreting an RdCVF are encapsulated. In some embodiments, cells are encapsulated within a semipermeable membrane that allows diffusion of RdCVF through the membrane. More information related to encapsulated cells and encapsulated cell implants is found in Sieving et al. (Proc Natl Acad Sci USA, (2006) 103(10):3896-901); U.S. Pat. Nos. 7,115,257 and 7,820,195; and PCT Publication No. WO2011044216. In some embodiments of the invention, encapsulated cells that express an RdCVF protein are delivered to an animal.

In some embodiments, encapsulated cells are implanted into a mammal, e.g., implanted in the eye, brain or olfactory region. In some embodiments, encapsulated cells are retinal pigment epithelial cells, e.g., ARPE-19 (available from ATCC, Manassas, VA). In some embodiments, encapsulated cells are used to deliver RdCVF to the eye, e.g., to the back of the eye.

In some embodiments, an encapsulated cell implant of the invention is comprised of cells that are encapsulated in a section of semi-permeable hollow fiber membrane and the cells have been genetically modified to produce an RdCVF. In some embodiments, an encapsulated cell implant has a suture loop at one end to anchor it to the sclera in the vitreo-retinal body inside the eye. In some embodiments, an encapsulated cell implant is 3, 4, 5, 6, 7, 8, 9 or 10 mm in length.

### RdCVF Protein Secretion and Expression

Nucleic acids and viral vectors of the invention can be used to express, produce and/or secrete an RdCVF from a cell. This expression, production and/or secretion can occur in vitro, in vivo or ex vivo.

Some embodiments of the invention provide methods of secreting an RdCVF protein from a cell comprising administering to the cell a nucleic acid and/or a viral vector of the invention. In some embodiments, the cell can be a mammalian cell, a human cell, an ocular cell, a retinal pigment epithelial (RPE) cell, a rod cell or a cone cell.

Some embodiments of the invention utilize vertebrate or mammalian cells. Examples of useful mammalian host cell lines are a monkey kidney CVI cell line transformed by SV40 (e.g., COS-7, ATCC CRL 1651); human embryonic kidney line (e.g., 293 or 293T cells including either cell line subcloned for growth in suspension culture, Graham et al., J. Gen Virol. 36:59 (1977) such as 293 Freestyle (Invitrogen, Carlsbad, CA)) or 293FT; baby hamster kidney cells (e.g., BHK, ATCC CCL 10); Chinese hamster ovary cells (CHO cells); Chinese hamster ovary cells/-DHFR (e.g., CHO, Urlaub et al., Proc. Natl. Acad. Sci. USA 77:4216 (1980)); mouse sertoli cells (e.g., TM4, Mather, Biol. Reprod. 23:243-251 (1980)); monkey kidney cells (e.g., CVI ATCC CCL 70); African green monkey kidney cells (e.g., VERO-76, ATCC CRL-1587); human cervical carcinoma cells (e.g., HELA, ATCC CCL 2); canine kidney cells (e.g., MOCK, ATCC CCL 34); CF2TH cells; buffalo rat liver cells (e.g., BRL 3A, ATCC CRL 1442); human lung cells (e.g., W138, ATCC CCL 75); human liver cells (e.g., Hep G2, HB 8065); mouse mammary tumor cells (e.g., MMT 060562, ATCC CCL51); TRI cells (Mather et al., Annals N.Y. Acad. Sci. 383:44-68 (1983)); MRC 5 cells; ARPE-19 cells (ATCC) and FS4 cells. In embodiments, the invention utilizes an immortalized human cell line, such as an immortalized human embryonic cell. In embodiments, the cell is an immortalized human embryonic kidney cell (HEK). In embodiments, the cell is HEK293 and variants, such as HEK293F and HEK293FT.

In embodiments, the cell expressing the fusion protein of the invention is a yeast cell, for example, Pichia pastoris, also known as Komagataella phaffi.

In some embodiments, a cell is selected from the group consisting of a 293 cell, a CHO cell, a PerC6 cell, a Vero cell, a BHK cell, a HeLa cell, a COS cell, a MDCK cell, a 3T3 cell or a WI38.

Some embodiments of the invention provide an isolated cell comprising a nucleic acid of the invention. In some embodiments, the nucleic acid is integrated into the cellular genome/DNA.

The invention also includes methods for producing an RdCVF protein comprising culturing a cell under conditions that allow for expression and secretion of the RdCVF protein and isolating the RdCVF protein from the cell culture, wherein the cell comprises a nucleic acid of the invention that codes for and allows the expression of the RdCVF protein, e.g., secretion of an RdCVF protein. In some embodiments, the nucleic comprises a nucleotide sequence comprises a coding sequence for an RdCVF protein, wherein the RdCVF coding sequence comprises a recoded sequence. The RdCVF protein can be an RdCVF 1 or 2 short form protein. In some embodiments, these methods further comprise purification of the RdCVF protein from the cell and/or culture supernatant.

The invention also includes an RdCVF protein expressed by a cell from a nucleic acid of the invention. The invention also provides secreted forms of RdCVF proteins of the invention and compositions comprising a secreted RdCVF protein of the invention.

In some embodiments, an RdCVF protein expressed from a cell is purified to at least 90%, at least 93%, at least 95%, at least 98%, at least 99.5% or at least 99.9% pure in relation to total protein.

### Compositions, Formulations and Preparations

Some embodiments of the invention provide compositions, formulations or preparations, e.g., pharmaceutical compositions, containing a nucleic acid of the invention, a vector of the invention, a RdCVF protein of the invention, or any combination thereof.

Formulations (e.g., for injection) are generally, but not necessarily, biocompatible solutions of the active ingredient, e.g., comprising Hank's solution, Ringer's solution or phosphate buffered saline. In some embodiments, a formulation or pharmaceutical composition comprises one or more of the following: citrate, NaCl, potassium chloride (KCl), calcium chloride dihydrate (CaCl2·2H2O), magnesium chloride hexahydrate (MgCl2·6H2O), sodium acetate trihydrate (CH3CO2Na·3H2O), sodium citrate dihydrate (C6H5O7Na3·2H2O), sucrose, sodium hydroxide and/or hydrochloric acid (to adjust pH) and water. The preceding list includes some molecules that are listed as particular hydrates, e.g., dihydrate, trihydrate, hexahydrate, etc. It is understood that various hydrates of these compounds can be used in the invention and the invention is not limited to these particular hydrate forms of the listed molecules. In some embodiments, a formulation or pharmaceutical composition comprises one or more ingredients selected from the group consisting of histidine, MgCl2, trehalose, a polysorbate, polysorbate 20, NaCl, sucrose, arginine and proline. In some embodiments, a formulation comprises one or more of the following: histidine; α, α-trehalose dehydrate; MgCl2; a polysorbate such as polysorbate 20; and NaCl. In some embodiments, a formulation or pharmaceutical composition comprises one or more of the following: phosphate buffered saline (PBS) and pluronic F-68. In some embodiments, pluronic F-68 concentration can be 0.0001%, 0.001%, 0.005%, 0.01% or 0.1%.

Examples of suitable formulations and formulatory methods for a desired mode of administration may be found in Remington's Pharmaceutical Sciences, latest edition, Mack Publishing Co., Easton, PA and in U.S. Pat. No. 7,208,577.

In some embodiments, a composition for use in vivo contains a “carrier” or a “pharmaceutically acceptable carrier”. The term “carrier” refers to a diluent, adjuvant, excipient, or vehicle with which a nucleic acid, vector or protein of the invention is administered. The term “carrier’ includes, but is not limited to, either solid or liquid material, which may be inorganic or organic and of synthetic or natural origin, with which an active component(s) of the composition is mixed or formulated to facilitate administration to a subject. Any other materials customarily employed in formulating a pharmaceutical are suitable. In embodiments, pharmaceutical carriers differ from typical solutions and suspensions in that they are specifically prepared for use in vivo to exclude substances that may be harmful to the host to whom the composition is administered (e.g., removal of bacterial toxins).

Examples of suitable liquid carriers include water and aqueous solutions containing oxygenated organic compounds such as ethanol. Buffers and other materials normally present in pharmaceutical preparations, such as flavoring and suspending agents, can also be present. In general, a suitable oil(s), saline, aqueous dextrose (glucose), and related sugar solutions and glycols such as propylene glycol or polyethylene glycols are typically suitable carriers for parenteral solutions. In some embodiments, solutions for parenteral administration contain a water-soluble salt of the active ingredient, suitable stabilizing agents, and if desirable or necessary, buffer substances. Antioxidizing agents such as sodium bisulfite, sodium sulfite, or ascorbic acid, either alone or combined, can be used as stabilizing agents. Also used are citric acid and its salts and sodium EDTA. In addition, parenteral solutions can contain preservatives, such as benzalkonium chloride, methyl- or propyl-paraben and chlorobutanol.

In embodiments, carriers are carbohydrates, including but not limited to trehalose, mannitol, glutathione, xylitol, sucrose, lactose and sorbitol. In embodiments, the formulations of the disclosure include, natural or synthetic surfactants, for example, DPPC (1,2-Didecanoyl-sn-glycero-3-phosphocholine), DOPE (1,2-Dioleoyl-sn-glycero-3-phosphoethanolamine), DSPC (1,2-Distearoyl-sn-glycero-3-phosphocholinez 1,2-Distearoyl-sn-glycero-3-phosphocholine) and DOPC (1,2-Dioleoyl-sn-glycero-3-phosphocholine). In embodiments, the formulations of the invention include polyethylene glycol. In embodiments, the formulations of the invention include dextrans, such as cyclodextran. In embodiments, the formulations of the invention include cyclodextrin, tertiary amines and/or beta-cyclodextrin. In embodiments, the formulations of the invention include enhancers, such as bile salts. In embodiments, the formulations of the invention include cellulose and cellulose derivatives. In embodiments, the formulations of the invention include amino acids. In embodiments, the formulations of the invention include liposomes, microcapsules or microspheres, inclusion complexes, or other types of carriers is contemplated.

In embodiments, the formulations of the invention include wetting and/or emulsifying agents, and/or pH buffering agents. In embodiments, the formulations of the invention include a solubilizing agent and/or a local anesthetic such as lignocaine to ease pain at the site of the injection.

In some embodiments, a pharmaceutical preparation or composition of the invention comprises a (i) pharmaceutically acceptable carrier and (ii) a nucleic acid of the invention, a viral vector of the invention, an RdCVF protein of the invention or any combination thereof.

### Treatment, Administration and Delivery RdCVF Coding Sequences

It has been demonstrated that an RdCVF protein can promote cone photoreceptor cell survival in vitro and in vivo. For example, intraocular injections of the short form of human RdCVF1 (RdCVF1S) protein not only rescued cone cells from degeneration but also preserved their function in animal models of inherited retinal degeneration. (Yang et al. (Mol Therapy (2009) 17:787-795 and the supplemental material). Expression of endogenous RdCVF1 is mainly restricted to the retina (Léveillard et al. (2004) Nature Genetics 36:755-759).

In embodiments, the disclosure provides methods of preserving ocular rod and/or cone cells comprising administering to the eye of a mammal a nucleic acid of the invention, a vector, e.g., viral vector, of the invention, an RdCVF protein of the invention, a pharmaceutical composition of the invention or a combination thereof. In embodiments, the term “preserved” means maintaining the function of the ocular rod cells, for example, the function is maintained at 100%, 90%, 85%, 80%, 75%, 70%, 65%, 60%, 55%, 50%, 45%, 40%, 35%, 30%, 25%, 20%, 15%, 10% or 5% of function of healthy ocular rod cells. In some embodiments, a viral vector and/or nucleic acid of the invention is administered by subretinal injection, intravitreal injection, injection to the intraanterior chamber of the eye, subconjunctival injection, subtenon injection or any combination thereof. In some embodiments, the mammal to be treated is a domesticated mammal, such as a cat, dog and horse. In embodiments, the mammal to be treated is a human.

In embodiments, the disclosure provides methods of preserving ocular rod and/or cone cells comprising administering to the eye of a mammal a nucleic acid of the invention, a viral vector of the invention, an RdCVF protein of the invention, a pharmaceutical composition of the invention or a combination thereof. In embodiments, the term “preserved” means maintaining the function of the ocular rod and/or cone cells, for example, the function is maintained at 100%, 90%, 85%, 80%, 75%, 70%, 65%, 60%, 55%, 50%, 45%, 40%, 35%, 30%, 25%, 20%, 15%, 10% or 5% of function of healthy ocular cone cells. In some embodiments, a viral vector and/or nucleic acid of the invention is administered by subretinal injection, intravitreal injection, injection to the intraanterior chamber of the eye, subconjunctival injection, subtenon injection or any combination thereof. In some embodiments, the mammal to be treated is a domesticated mammal, such as a cat, dog and horse. In embodiments, the mammal to be treated is a human.

In some embodiments, the mammal to be treated suffers from an ocular disease, such as a retinal dystrophy, Stargardt's disease, retinitis pigmentosa, dry age-related macular degeneration (dry AMD), geographic atrophy (advanced stage of dry AMD), wet age-related macular degeneration (wet AMD), glaucoma/ocular hypertension, diabetic retinopathy, Bardet-Biedel syndrome, Bassen-Kornzweig syndrome, Best disease, choroidema, gyrate atrophy, congenital amaurosis, Refsum syndrome, Usher syndrome, thyroid related eye disease, Grave's disease, a disease associated with retinal pigmented epithelial cells, anterior segment disease, lens disease/cataracts, an eye cup disorder, or uveitis. In some embodiments, the preserved ocular rod cell does not contain a nucleic acid and/or viral vector of the invention. For example, the preserved ocular cell is not preserved through transduction of the preserved ocular cell itself.

The present disclosure further provides a method of preserving the function of ocular rod and/or cone cells comprising administering to the eye of a mammal a nucleic acid and/or viral vector of the invention, wherein the nucleic acid and/or the viral vector is administered by subretinal injection and the rod cells and/or cones cells are preserved at a site at least 1 mm, at least 2 mm, at least 3 mm, at least 5 mm, at least 7 mm, at least 10 mm, at least 15 mm, at least 20 mm, at least 25 mm, or at least 2 cm from the site of the subretinal injection. For example and not wishing to be bound by theory, the cells transduced with the nucleic acid or viral vector at the subretinal injection site expresses and/or secrete an RdCVF protein, which can provide an ocular rod and/or cone preserving effect at a site distant to the transduced cell or injection site.

It is understood that when introduction or administration of a nucleic acid or vector encoding an RdCVF protein is disclosed, that the disclosure also provides introducing or administering the RdCVF protein itself. It is understood that when introduction of an RdCVF protein is disclosed, the invention also discloses introducing a nucleic acid or vector encoding an RdCVF protein.

In some embodiments, compositions of the invention are administered locally or systemically. Useful routes of administration are described herein and known in the art. Methods of introduction or administration include, but are not limited to, intradermal, intramuscular, intraperitoneal, intravenous, subcutaneous, intranasal, intratracheal, topical, inhaled, transdermal, rectal, parenteral routes, epidural, intracranial, into the brain, intraventricular, subdural, intraarticular, intrathecal, intracardiac, intracoronary, intravitreal, subretinal, intraanterior chamber, suprachoroidal of the eye, locally on the cornea, subconjunctival, subtenon injection, by applying eyedrops, oral routes, via balloon catheter, via stent or any combinations thereof. Systemic administration may be, but is not limited to, by intravenous or intra-arterial injection or by transmucosal, subcutaneous, transdermal and/or intraperitoneal delivery.

In some embodiments, e.g., comprising administration to the eye, an RdCVF1-S or RdCVF2-S encoding vector or nucleic acid of the invention is administered about once every week, month, 2 months, 3 months, 6 months, 9 months, year, 18 months, 2 years, 30 months, 3 years, 5 years, 10 years or as needed. In some embodiments, e.g., comprising administration to the eye, an RdCVF encoding vector or nucleic acid of the invention is administered from about every 1 to 4 weeks, about every 4 to 8 weeks, about every 1 to 4 months, about every 3 to 6 months, about every 4 to 8 months, about every 6 to 12 months, about every 9 to 15 months, about every 12 to 18 months, about every 15 to 21 months, about every 18 to 24 months, about every 1 to 2 years, about every 1.5 to 3 years, about every 2 to 4 years, about every 3 to 5 years, about every 5 to 7 years, about every 7 to 10 years or about every 10 to 20 years. It is expected that administration of a vector coding for an RdCVF protein would be less frequent than administration of the RdCVF protein itself. In some embodiments of the invention, a pharmaceutical preparation comprises a vector encoding an RdCVF protein of the invention and the pharmaceutical preparation is administered only once to the subject.

In some embodiments, an RdCVF1-S or RdCVF2-S encoding vector or nucleic acid of the invention is administered by intravitreal or subretinal injection to a human eye. In some embodiments, about 15 g to about 5 mg; about 15 g to about 500 g; about 100 g to about 900 g; about 300 g to about 700 g; about 500 g to about 1 mg; about 1 mg to about 5 mg; about 1 mg; or about 500 g of an RdCVF protein is administered by intravitreal or subretinal injection to a human eye.

In some embodiments, an RdCVF1-S or RdCVF2-S encoding vector or nucleic acid of the invention is administered by subretinal injection or intravitreal injection. In some embodiments, about 5×108 to about 1×109; about 5×108 to about 7.5×108; about 7.5×108 to about 1×109; about 6×108 to about 9×108; about 7×108 to about 8×108; about 5×108; about 6×108; about 7×108; about 8×108; about 9×108; or about 1×109; or about 1×1010; or about 1×1011; or about 1×1012 vector genome copy (GC) number of an AAV vector is administered by subretinal injection. In some embodiments, about 5×108 to about 1×1010; about 5×108 to about 5×109; about 5×108 to about 2×109; about 2×109 to about 5×109; about 5×109 to about 1×1010; about 5×108 to about 1×109; about 1×109 to about 3×109; about 3×109 to about 6×109; about 6×109 to about 1×1010; about 1×109 to about 1×1010; about 1×1010 to about 1×1011; or 1×1011 to about 1×1012; or 1×1012 to about 5×1012 GC of an AAV vector is administered by intravitreal injection. In some embodiments, about 5×108 to about 1×1010; about 5×108 to about 5×109; about 5×108 to about 2×109; about 2×109 to about 5×109; about 5×109 to about 1×1010; about 5×108 to about 1×109; about 1×109 to about 3×109; about 3×109 to about 6×109; about 6×109 to about 1×1010; about 1×109 to about 1×1010; about 1×1010 to about 1×1011; 1×1011 to about 1×1012; 1×1012 to about 1×1013; 1×1013 to about 1×1014; 1×1014 to about 5×1014 GC of an AAV vector is administered by intrathecal injection. In some embodiments, about 5×108 to about 1×1010; about 5×108 to about 5×109; about 5×108 to about 2×109; about 2×109 to about 5×109; about 5×109 to about 1×1010; about 5×108 to about 1×109; about 1×109 to about 3×109; about 3×109 to about 6×109; about 6×109 to about 1×1010; about 1×109 to about 1×1010; about 1×1010 to about 1×1011; 1×1011 to about 1×1012; 1×1012 to about 1×1013; 1×1013 to about 1×1014; 1×1014 to about 1×1015 GC of an AAV vector comprising the RdCVF1L or RdCVF2L protein is administered by intravenous injection. It is understood that the amount of AAV vector is sometimes measured in transducing units or in GC number. GC numbers are typically between 25-300 times higher than when the same AAV vector sample is measured for transducing units.

In some embodiments, this disclosure provides a method for treating a condition in a mammalian subject in need thereof, comprising administering to the subject an effective amount of the fusion protein, wherein the condition is retinal dystrophy, Stargardt's disease, retinitis pigmentosa, dry age-related macular degeneration (dry AMD), geographic atrophy (advanced stage of dry AMD), wet age-related macular degeneration (wet AMD), glaucoma with or without ocular hypertension, diabetic retinopathy, Bardet-Biedel syndrome, Bassen-Kornzweig syndrome, Best disease, choroidema, gyrate atrophy, congenital amaurosis, Refsum syndrome, Usher syndrome, thyroid related eye disease, Grave's disease, a disease associated with retinal pigmented epithelial cells, anterior segment disease, lens disease/cataracts, an eye cup disorder, uveitis, Alzheimer's disease, Huntington's disease, Parkinson's disease, or an olfactory disease. In embodiments, the condition is an ocular condition and the administration is subretinal injection or intravitreal injection.

In some embodiments, this disclosure provides a method of protecting ocular photoreceptor cells in a subject in need thereof, comprising administering to an eye of the subject an effective amount of the fusion protein. In some embodiments, the administration is subretinal injection or intravitreal injection. In some embodiments, the subject is a human subject.

### DESCRIPTION OF THE SEQUENCES

Shuttle AAV vector construct
nucleotide sequence encoding hIgk(+Asp).hRdCVF1S
(from Left ITR to Right ITR):
SEQ ID NO: 1
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGCA
CGCGTGGAGCTAGTTATTAATAGTAATCAATTACGGGGTCATTAGTTCAT
AGCCCATATATGGAGTTCCGCGTTACATAACTTACGGTAAATGGCCCGCC
TGGCTGACCGCCCAACGACCCCCGCCCATTGACGTCAATAATGACGTATG
TTCCCATAGTAACGTCAATAGGGACTTTCCATTGACGTCAATGGGTGGAG
TATTTACGGTAAACTGCCCACTTGGCAGTACATCAAGTGTATCATATGCC
AAGTACGCCCCCTATTGACGTCAATGACGGTAAATGGCCCGCCTGGCATT
ATGCCCAGTACATGACCTTATGGGACTTTCCTACTTGGCAGTACATCTAC
GTATTAGTCATCGCTATTACCATGGTGATGCGGTTTTGGCAGTACATCAA
TGGGCGTGGATAGCGGTTTGACTCACGGGGATTTCCAAGTCTCCACCCCA
TTGACGTCAATGGGAGTTTGTTTTGCACCAAAATCAACGGGACTTTCCAA
AATGTCGTAACAACTCCGCCCCATTGACGCAAATGGGCGGTAGGCGTGTA
CGGTGGGAGGTCTATATAAGCAGAGCTCGTTTAGTGAACCGTCAGATCGC
CTGGAGACGCCATCCACGCTGTTTTGACCTCCATAGAAGACACCGGGACC
GATCCAGCCTCCGCGGATTCGAATCCCGGCCGGGAACGGTGCATTGGAAC
GCGGATTCCCCGTGCCAAGAGTGACGTAAGTACCGCCTATAGAGTCTATA
GGCCCACAAAAAATGCTTTCTTCTTTTAATATACTTTTTTGTTTATCTTA
TTTCTAATACTTTCCCTAATCTCTTTCTTTCAGGGCAATAATGATACAAT
GTATCATGCCTCTTTGCACCATTCTAAAGAATAACAGTGATAATTTCTGG
GTTAAGGCAATAGCAATATTTCTGCATATAAATATTTCTGCATATAAATT
GTAACTGATGTAAGAGGTTTCATATTGCTAATAGCAGCTACAATCCAGCT
ACCATTCTGCTTTTATTTTATGGTTGGGATAAGGCTGGATTATTCTGAGT
CCAAGCTAGGCCCTTTTGCTAATCATGTTCATACCTCTTATCTTCCTCCC
ACAGCTCCTGGGCAACGTGCTGGTCTGTGTGCTGGCCCATCACTTTGGCA
AAGAATTGGGATTCGAACATCGATTGAATTCGAGCCACCATGGACATGAG
GGTCCCTGCTCAGCTCCTGGGGCTCCTGCTGCTCTGGCTCTCAGGTGCCA
GATGTGATGCCAGCCTGTTCAGCGGCCGGATCCTGATCAGGAACAACAGC
GACCAGGACGAGCTGGACACCGAGGCCGAAGTGAGCAGGAGGCTGGAGAA
CAGACTGGTGCTGCTGTTCTTTGGCGCCGGAGCCTGCCCTCAGTGCCAGG
CCTTCGTGCCCATCCTGAAGGATTTCTTTGTGCGGCTGACCGACGAGTTC
TACGTGCTGAGAGCCGCCCAGCTGGCCCTGGTGTATGTGAGCCAGGACAG
CACCGAGGAGCAGCAGGACCTGTTCCTGAAGGACATGCCCAAGAAGTGGC
TGTTCCTGCCCTTCGAGGACGACCTGCGGAGATGAGCTAGCACCGGTTGT
ACAAGTCAAGCGGCCAACCCTCCCTAGATCTACGGGTGGCATCCCTGTGA
CCCCTCCCCAGTGCCTCTCCTGGCCCTGGAAGTTGCCACTCCAGTGCCCA
CCAGCCTTGTCCTAATAAAATTAAGTTGCATCATTTTGTCTGACTAGGTG
TCCTTCTATAATATTATGGGGTGGAGGGGGGTGGTATGGAGCAAGGGGCA
AGTTGGGAAGACAACCTGTAGGGCCTGCGGGGTCTATTGGGAACCAAGCT
GGAGTGCAGTGGCACAATCTTGGCTCACTGCAATCTCCGCCTCCTGGGTT
CAAGCGATTCTCCTGCCTCAGCCTCCCGAGTTGTTGGGATTCCAGGCATG
CATGACCAGGCTCAGCTAATTTTTGTTTTTTTGGTAGAGACGGGGTTTCA
CCATATTGGCCAGGCTGGTCTCCAACTCCTAATCTCAGGTGATCTACCCA
CCTTGGCCTCCCAAATTGCTGGGATTACAGGCGTGAACCACTGCTCCCTT
CCCTGTCCTTCTGATTTTGTAGGTAACCACGTGCGGACCGAGCGGCCGCA
GGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGG
GCGGCCTCAGTGAGCGAGCGAGCGCGCAGCTGCCTGCAGG
Amino acid sequence of hIgk(+Asp).
hRdCVF1S (Human RdCVF1S and Human Igk signal
sequence (+Asp)):
SEQ ID NO: 2
MDMRVPAQLLGLLLLWLSGARCDASLFSGRILIRNNSDQDELDTEAEVSR
RLENRLVLLFFGAGACPQCQAFVPILKDFFVRLTDEFYVLRAAQLALVYV
SQDSTEEQQDLFLKDMPKKWLFLPFEDDLRR
Shuttle AAV vector construct
nucleotide sequence encoding hIgk(+Asp).hRdCVF1S.
hIgG4-Fc (from Left ITR to Right ITR):
SEQ ID NO: 3
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCGCA
CGCGTGGAGCTAGTTATTAATAGTAATCAATTACGGGGTCATTAGTTCAT
AGCCCATATATGGAGTTCCGCGTTACATAACTTACGGTAAATGGCCCGCC
TGGCTGACCGCCCAACGACCCCCGCCCATTGACGTCAATAATGACGTATG
TTCCCATAGTAACGTCAATAGGGACTTTCCATTGACGTCAATGGGTGGAG
TATTTACGGTAAACTGCCCACTTGGCAGTACATCAAGTGTATCATATGCC
AAGTACGCCCCCTATTGACGTCAATGACGGTAAATGGCCCGCCTGGCATT
ATGCCCAGTACATGACCTTATGGGACTTTCCTACTTGGCAGTACATCTAC
GTATTAGTCATCGCTATTACCATGGTGATGCGGTTTTGGCAGTACATCAA
TGGGCGTGGATAGCGGTTTGACTCACGGGGATTTCCAAGTCTCCACCCCA
TTGACGTCAATGGGAGTTTGTTTTGCACCAAAATCAACGGGACTTTCCAA
AATGTCGTAACAACTCCGCCCCATTGACGCAAATGGGCGGTAGGCGTGTA
CGGTGGGAGGTCTATATAAGCAGAGCTCGTTTAGTGAACCGTCAGATCGC
CTGGAGACGCCATCCACGCTGTTTTGACCTCCATAGAAGACACCGGGACC
GATCCAGCCTCCGCGGATTCGAATCCCGGCCGGGAACGGTGCATTGGAAC
GCGGATTCCCCGTGCCAAGAGTGACGTAAGTACCGCCTATAGAGTCTATA
GGCCCACAAAAAATGCTTTCTTCTTTTAATATACTTTTTTGTTTATCTTA
TTTCTAATACTTTCCCTAATCTCTTTCTTTCAGGGCAATAATGATACAAT
GTATCATGCCTCTTTGCACCATTCTAAAGAATAACAGTGATAATTTCTGG
GTTAAGGCAATAGCAATATTTCTGCATATAAATATTTCTGCATATAAATT
GTAACTGATGTAAGAGGTTTCATATTGCTAATAGCAGCTACAATCCAGCT
ACCATTCTGCTTTTATTTTATGGTTGGGATAAGGCTGGATTATTCTGAGT
CCAAGCTAGGCCCTTTTGCTAATCATGTTCATACCTCTTATCTTCCTCCC
ACAGCTCCTGGGCAACGTGCTGGTCTGTGTGCTGGCCCATCACTTTGGCA
AAGAATTGGGATTCGAACATCGATTGAATTCGAGCCACCATGGACATGAG
GGTCCCTGCTCAGCTCCTGGGGCTCCTGCTGCTCTGGCTCTCAGGTGCCA
GATGTGATGCCAGCCTGTTCAGCGGCCGGATCCTGATCAGGAACAACAGC
GACCAGGACGAGCTGGACACCGAGGCCGAAGTGAGCAGGAGGCTGGAGAA
CAGACTGGTGCTGCTGTTCTTTGGCGCCGGAGCCTGCCCTCAGTGCCAGG
CCTTCGTGCCCATCCTGAAGGATTTCTTTGTGCGGCTGACCGACGAGTTC
TACGTGCTGAGAGCCGCCCAGCTGGCCCTGGTGTATGTGAGCCAGGACAG
CACCGAGGAGCAGCAGGACCTGTTCCTGAAGGACATGCCCAAGAAGTGGC
TGTTCCTGCCCTTCGAGGACGACCTGCGGAGAGGACGACCCCCATGCCCA
TCATGCCCAGCACCTGAGTTCCTGGGGGGACCATCAGTCTTCCTGTTCCC
CCCAAAACCCAAGGACACTCTCATGATCTCCCGGACCCCTGAGGTCACGT
GCGTGGTGGTGGACGTGAGCCAGGAAGACCCCGAGGTCCAGTTCAACTGG
TACGTGGATGGCGTGGAGGTGCATAATGCCAAGACAAAGCCGCGGGAGGA
GCAGTTCAACAGCACGTACCGTGTGGTCAGCGTCCTCACCGTCCTGCACC
AGGACTGGCTGAACGGCAAGGAGTACAAGTGCAAGGTCTCCAACAAAGGC
CTCCCGTCCTCCATCGAGAAAACCATCTCCAAAGCCAAAGGGCAGCCCCG
AGAGCCACAGGTGTACACCCTGCCCCCATCCCAGGAGGAGATGACCAAGA
ACCAGGTCAGCCTGACCTGCCTGGTCAAAGGCTTCTACCCCAGCGACATC
GCCGTGGAGTGGGAGAGCAATGGGCAGCCGGAGAACAACTACAAGACCAC
GCCTCCCGTGCTGGACTCCGACGGCTCCTTCTTCCTCTACAGCAGGCTAA
CCGTGGACAAGAGCAGGTGGCAGGAGGGGAATGTCTTCTCATGCTCCGTG
ATGCATGAGGCTCTGCACAACCACTACACACAGAAGAGCCTCTCCCTGTC
TCTGGGTAAATAATGAGCTAGCACCGGTTGTACAAGTCAAGCGGCCAACC
CTCCCTAGATCTACGGGTGGCATCCCTGTGACCCCTCCCCAGTGCCTCTC
CTGGCCCTGGAAGTTGCCACTCCAGTGCCCACCAGCCTTGTCCTAATAAA
ATTAAGTTGCATCATTTTGTCTGACTAGGTGTCCTTCTATAATATTATGG
GGTGGAGGGGGGTGGTATGGAGCAAGGGGCAAGTTGGGAAGACAACCTGT
AGGGCCTGCGGGGTCTATTGGGAACCAAGCTGGAGTGCAGTGGCACAATC
TTGGCTCACTGCAATCTCCGCCTCCTGGGTTCAAGCGATTCTCCTGCCTC
AGCCTCCCGAGTTGTTGGGATTCCAGGCATGCATGACCAGGCTCAGCTAA
TTTTTGTTTTTTTGGTAGAGACGGGGTTTCACCATATTGGCCAGGCTGGT
CTCCAACTCCTAATCTCAGGTGATCTACCCACCTTGGCCTCCCAAATTGC
TGGGATTACAGGCGTGAACCACTGCTCCCTTCCCTGTCCTTCTGATTTTG
TAGGTAACCACGTGCGGACCGAGCGGCCGCAGGAACCCCTAGTGATGGAG
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGCTGCCTGCAGG
Amino acid sequence of hIgk(+Asp).
hRdCVF1S.hIgG4-Fc:
SEQ ID NO: 4
MDMRVPAQLLGLLLLWLSGARCDASLFSGRILIRNNSDQDELDTEAEVSR
RLENRLVLLFFGAGACPQCQAFVPILKDFFVRLTDEFYVLRAAQLALVYV
SQDSTEEQQDLFLKDMPKKWLFLPFEDDLRRGRPPCPSCPAPEFLGGPSV
FLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK
Amino Acid sequence of Human
Igk secretory signal peptide
(+Asp):
SEQ ID NO: 5
MDMRVPAQLLGLLLLWLSGARCD
Amino Acid sequence of hRdCVFIS:
SEQ ID NO: 6
ASLESGRILIRNNSDQDELDTEAEVSRRLENRLVLLFFGAGACPQCQAFV
PILKDFFVRLTDEFYVLRAAQLALVYVSQDSTEEQQDLFLKDMPKKWLFL
PFEDDLRR
Amino Acid sequence of hIgG4-Fc:
SEQ ID NO: 7
GRPPCPSCPAPEFLGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK
Amino Acid sequence of Human Igk
secretory signal peptide:
SEQ ID NO: 8
MDMRVPAQLLGLLLLWLSGARC
Amino Acid sequence of Human Igk
secretory signal sequence I:
SEQ ID NO: 9
METDTLLWVLLLWVPGSTGDDIV
Amino acid sequence of hIgk(I).
hRdCVF1S (Human RdCVFIS
and Human Igk signal sequence I):
SEQ ID NO: 10
METDTLLWVLLLWVPGSTGDDIVASLFSGRILIRNNSDQDELDTEAEVSR
RLENRLVLLFFGAGACPQCQAFVPILKDFFVRLTDEFYVLRAAQLALVYV
SQDSTEEQQDLFLKDMPKKWLFLPFEDDLRR
Amino acid sequence of hIgk(I).
hRdCVF1S (Human RdCVFIS
and Human Igk signal sequence (-Asp)):
SEQ ID NO: 11
MDMRVPAQLLGLLLLWLSGARCASLESGRILIRNNSDQDELDTEAEVSRR
LENRLVLLFFGAGACPQCQAFVPILKDFFVRLTDEFYVLRAAQLALVYVS
QDSTEEQQDLFLKDMPKKWLFLPFEDDLRR
Amino acid sequence of hIgk(I).
hRdCVF1S.hIgG4-Fc:
SEQ ID NO: 12
METDTLLWVLLLWVPGSTGDDIVASLESGRILIRNNSDQDELDTEAEVSR
RLENRLVLLFFGAGACPQCQAFVPILKDFFVRLTDEFYVLRAAQLALVYV
SQDSTEEQQDLFLKDMPKKWLFLPFEDDLRRGRPPCPSCPAPEFLGGPSV
FLFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLGK
Amino acid sequence of hIgk(-Asp).
hRdCVF1S.hIgG4-Fc:
SEQ ID NO: 13
MDMRVPAQLLGLLLLWLSGARCASLFSGRILIRNNSDQDELDTEAEVSRR
LENRLVLLFFGAGACPQCQAFVPILKDFFVRLTDEFYVLRAAQLALVYVS
QDSTEEQQDLFLKDMPKKWLFLPFEDDLRRGRPPCPSCPAPEFLGGPSVF
LFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKG
QPREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKSL
SLSLGK

The present disclosure has been described with respect to representative examples that are to be considered illustrative embodiments that do not limit the scope of the disclosure which is defined solely by the claims. All references to publications, including scientific publications, treatises, textbooks, patent applications and issued patents are hereby incorporated by reference for all purposes.

### EXAMPLES

### Example 1. Plasmid Cloning of AAV Transfer Vector Constructs

The recoded open reading frames for the short form of human rod derived cone viability factor (hRdCVF1S) and hRdCVF1S fused to human IgG4-Fc (hRdCVF1S.hIgG4-Fc), both containing human Igk secretory signal sequence, were synthesized as eBlocks and cloned into an AAV transfer vector by Gibson assembly, creating plasmids pAAV.hRdCVF1S and pAAV.hRdCVF1S.hIgG4-Fc, respectively. The sequence identities of these constructs were confirmed by sanger sequencing and prepped for rAAV production.

### Example 2. rAAV8 Production and Purification

pAAV.hRdCVF1S and pAAV.hRdCVF1S.hIgG4-Fc were packaged as rAAV8 gene therapy vectors using a triple transfection protocol.

HEK293FT cells were cultured in DMEM with GlutaMAX and supplemented with non-essential amino acids, sodium pyruvate, and 10% FBS and antibiotics (100 μg/mL penicillin and 50 μg/mL streptomycin). At ˜72 hr before transfection, cells were counted and seeded in 15-cm dishes at 6.0×106 cells per dish.

On the day of transfection, the growth media was removed and replaced with fresh media. The cells were then transfected with PEI (1 μg/μL) using a PEI:DNA mass ratio of 2. Each 15-cm dish was transfected with 50.75 μg (0.35 μg/cm2) plasmid DNA in a 1:1:1 molar ratio of pRC8c, pHelper and either of the pAAV shuttle vectors described above in a 1:1:1 molar ratio.

After ˜72 hours, the cells were scraped and the cells and media were collected and centrifuged at 1000×g for 5 min at 4° C. After centrifugation, the media was decanted and virus present in media was harvested by PEG precipitation by adding 5 g PEG 8000 and 0.3 g NaCl to each 50 mL of media, and incubating the media at 4° C. overnight. The cell pellet was resuspended in lysis buffer (dPBS+0.01% pluronic F68 and 200 mM NaCl) and lysed by 1× freeze and thaw followed by sonication. The lysate was clarified by centrifugation at 3000×g for 30 min at 4° C. and the supernatant was collected.

After incubation the PEG precipitation was centrifuged at 3000×g for 30 min at 4° C. and the media was discarded. The PEG pellet was resuspended in lysis buffer and combined with the clarified cell lysate. The lysate was then subjected to ultracentrifugation through an IDX density gradient. IDX was diluted in dPBS and underlaid in a 38.5 mL Quick-seal tub in the following order: 8 mL of 15% IDX with 1M NaCl, 6 mL of 25% IDX, 8 mL of 40% IDX and 5 mL of 60% IDX.

The clarified lysate was layered on top of the IDX gradient and centrifuged in a T70i rotor at 60,000 RPM (264,904×g) for 180 min at 10° C. After centrifugation, 4 mL of the 40% IDX layer was collected by puncturing the ultracentrifuge tubes with an 18-g needle just below the 40-60% IDX interface. The collected IDX containing AAV particles was buffer exchanged by repeated dilution with formulation buffer (dPBS with 0.001% pluronic F68) and concentration with Amicon Ultra-15 100K Centrifugal Filters. After final concentration, the AAV sample was sterilized by centrifugation through a 0.22 μM filter, aliquoted and stored at −80° C.

### Example 3. rAAV8 Genome Copy Titer Determination

Purified rAAV8.hRdCVF1S and rAAV8.hRdCVF1S.hIgG4-Fc titers, defined at genome copies (GC)/mL, were determined by qPCR using a standard curve method. A standard curve for calculating genome copy titer was generated by serial diluting a linearized pAAV.EGFP control vector from 2×108 to 2×103 copies/μL. Prior to performing qPCR, the rAAV samples were digested with DNase I to degrade any contaminating pAAV shuttle vector plasmid DNA. A 10 μL rAAV sample was diluted into 79 μL of nuclease-free H2O, 10 μL of 10×DNase reaction buffer and 1 μL of DNase I, and incubated at 37° C. for 20 min and 75° C. for 10 min. The DNase I digested sample was serial diluted in dPBS (1:10 and 1:100) and stored on ice for qPCR. Three 5 μL replicates of each sample dilution and standard curve concentration was added to 15 μL of SYBR Green Universal Master Mix containing 0.67 μM FWD and REV primers targeting the CMV promoter of the rAAV transgene expression cassette. qPCR was performed on a QuantStudio 6 Pro thermocycler using the following protocol: 10 min at 98° C., (melt for 15 sec at 95° C., anneal/extend for 60 sec at 60° C.)×40 cycles. A melt curve was performed to verify the specificity of PCR amplification. Titers were calculated according to the following equation: Titer (GC/mL)=quantity (calculated for standard curve)×10 (DNase I dilution)×2 (double stranded DNA standard)×dilution factor×1000 (convert μL to mL).

The titers of rAAV8.hRdCVF1S and rAAV8.hRdCVF1S.hIgG4-Fc were 4.63E+13 and 3.97E+13 GC/mL, respectively. The data suggest that under identical experimental conditions, the vector titers for both vectors were comparable and the additional coding sequence for IgG4-Fc did not compromise the production and the titer of the AAV vector.

### Example 4. Silver Stain Analysis for rAAV8.hIgk.hRdCVF1S and rAAV8.hIgκ.hRdCVF1S.hIgG4-Fc

An aliquot of rAAV8.hIgκ.hRdCVF1S and rAAV8.hIgκ.hRdCVF1S.hIgG4-Fc containing 2×10¹¹ genome copies was denatured in reducing sample buffer and electrophoresed on a 4-12% polyacrylamide gel. The gel was then stained using the Pierce silver staining kit according to the manufacturer's instructions. Briefly, the gel was washed 2×5 min in ultrapure H2O and fixed 2×15 min in 30% ethanol with 10% acetic acid. After fixation, the gel was washed 2×5 min in 10% ethanol and then 2×5 min in ultrapure H2O. The gel was then incubated in sensitizer solution (50 μL sensitizer in 25 mL ultrapure H2O) for 1 min and wash 2×1 min in ultrapure H2O. Next, the gel was stained (0.5 mL enhancer in 25 mL stain) for 30 min and washed 2×20 sec in ultrapure H2O. To develop, the gel was bathed in developer working solution (0.5 mL enhancer in 25 mL developer) until protein bands were evident. Development of the gel was stopped with 5% acetic acid for 10 min. The gel was washed in ultrapure H2O and imaged using a Gel Doc EZ imager.

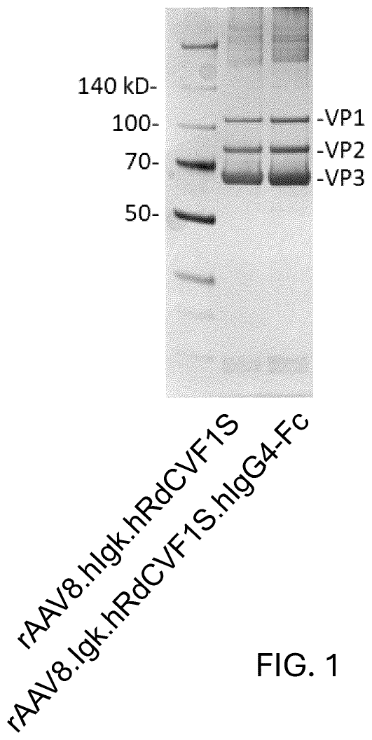

The presence of AAV8 particles was analyzed by silver staining of vector particle lysate. As shown in FIG. 1, the VP1, VP2 and VP3 capsid proteins of AAV8 were detected for both rAAV8.hIgk.hRdCVF1S and rAAV8.hIgk.hRdCVF1S.hIgG4-Fc vectors at comparable levels of density. The purity of both AAV vectors were similar.

### Example 5. rAAV8.hIgκ.hRdCVF1S and rAAV8.hIgκ.hRdCVF1S.hIgG4-Fc Gene Transfer in a Cell Model

Expression and secretion of hRdCVF1S and hRdCVF1S.hIgG4-Fc was evaluated in the HEK293FT cell line. HEK293FT cells were seeded in a 24 well plate at a seeding density of 4×10⁵ cells/well. The following day, cells were transduced with either rAAV8.hRdCVF1S or rAAV8.hRdCVF1S.hIgG4-Fc at an MOI of 100,000 (GC/cell), or not transduced as a control. After approximately 72 hrs., the media was collected, centrifuged at 16,000×g for 30 min and stored at −20° C. The cells were lysed with 100 μL ice cold RIPA buffer supplemented with Halt protease inhibitor cocktail, centrifuged at 16,000×g for 30 min and stored at −20° C. 22.75 microliters of each cell lysate and media sample was added to 8.75 μL of 4× sample buffer, and 3.5 μL 1M DTT, and denatured for 5 min at 95° C. Samples were electrophoresed on a 4-12% polyacrylamide gel and blotted to a nitrocellulose membrane. The nitrocellulose membrane was blocked in BSA for 1 hr at room temperature and incubated with primary rabbit anti-RdCVF (AD-10, 1:1000) antibodies in BSA over night at 4° C. After primary incubation, the membrane was washed 3×5 min in BSA, incubated in secondary HRP-conjugated goat anti-rabbit antibodies (1:5,000) in PBS-T for 1 hr at room temperature. Following secondary incubation, the membrane was washed 2×5 min in PBS-T and 1×5 min in PBS. The blot was developed with SuperSignal West Pico PLUS Chemiluminescent Substate and imaged on a BIO-RAD ChemiDoc MP imaging system.

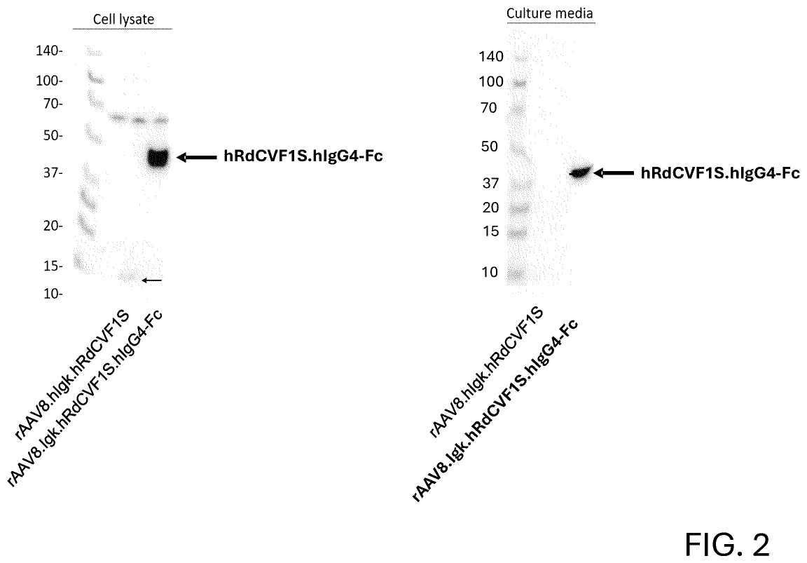

Proteins immunoreactive to anti-RdCVF antibodies were detected at ˜12 and ˜38 kDa (near the theoretical molecular weight of hRdCVF1S and hRdCVF1S.hIgG4-Fc, respectively) in cell lysates form HEK293FT cell cultures transduced with either rAAV8.hRdCVF1S or rAAV8.hRdCVF1S.hIgG4-Fc, respectively, but not in untransduced cells (FIG. 2, Left Panel). Even with a codon-optimized sequence and a human signal sequence, only very weak expression of hRdCVF1S was observed in the cell lysate transduced with rAAV8.hRdCVF1S vector (FIG. 2, Left Panel). A robust expression of hRdCVF1S.hIgG4-Fc was detected in the cell lysate transduced by rAAV8.hRdCVF1S.hIgG4-Fc vector under identical experimental conditions (FIG. 2, Left Panel). The secretion of hRdCVF1S was not detected in the cell culture supernatant transduced with rAAV8.hRdCVF1S at the level of sensitivity of the Western Blot (FIG. 2, Right Panel). In a great contrast, hRdCVF1S.hIgG4-Fc fusion protein was readily detected in the cell culture supernatant transduced with rAAV8.hRdCVF1S.hIgG4-Fc vector under identical experimental conditions (FIG. 2, Right Panel).

Together, the data indicate 1) human RdCVF1S was not efficiently expressed and its secretion could not be readily detected in the cell culture supernatant transduced by AAV vector even after the coding sequence was codon optimized for expression in mammalian cells and incorporation of a human Igk signal sequence to facilitate its secretion; and 2) by fusion with human IgG4-Fc and incorporation of a human Igk secretory signal sequence, expression and secretion of RdCVF1S were dramatically increased and could be readily detected in the cell lysate and cell culture supernatant transduced with AAV vector under the identical experimental conditions. The efficient expression and secretion of human RdCVF1S were likely resulted from the significant reduction of the hydrophobicity of the fusion protein.

1. A fusion protein comprising a first N-terminal signal peptide, a second peptide C-terminal to the signal peptide, and a third peptide C-terminal to the second peptide;
wherein the second peptide or the third peptide is an RdCVF-short peptide and the other is an immunoglobulin constant region.

2. The fusion protein of claim 1, wherein the second peptide is an RdCVF-short peptide, and the third peptide sequence is an immunoglobulin constant region.

3. The fusion protein of claim 1, wherein the second peptide is an immunoglobulin constant region, and the third peptide is an RdCVF-short peptide.

4. The fusion protein of claim 1, wherein the signal peptide is a human signal peptide.

5. The fusion protein of claim 1, wherein the signal peptide is an immunoglobulin kappa chain (Igk) signal peptide, a human growth hormone (HGH), a brain-derived neurotrophic factor (BDNF), an insulin growth factor 1 (IGF-1), a β-glucuronidase (GUSB), or an albumin signal peptide.

6-34. (canceled)

35. A nucleic acid comprising a nucleotide sequence encoding a fusion protein comprising a first N-terminal signal peptide, a second peptide C-terminal to the signal peptide, and a third peptide C-terminal to the second peptide sequence;
wherein the second peptide or the third peptide sequence is an RdCVF-short peptide sequence and the other is an immunoglobulin constant region sequence.

36-71. (canceled)

72. An expression vector comprising the nucleic acid of claim 35 operatively linked to a control sequence.

73-80. (canceled)

81. A cell comprising the fusion protein of claim 1;
a nucleic acid comprising a nucleotide sequence encoding the fusion protein; or
an expression vector comprising the nucleic acid operatively linked to a control sequence.

82. A pharmaceutical composition comprising: (i) the fusion protein of claim 1;
a nucleic acid comprising a nucleotide sequence encoding the fusion protein;
an expression vector comprising the nucleic acid operatively linked to a control sequence; and/or
a cell comprising the fusion protein, the nucleic acid, or the expression vector;
and (ii) a pharmaceutically acceptable carrier.

83. A method for treating a condition in a mammalian subject in need thereof, comprising administering to the subject an effective amount of the fusion protein of claim 1;
a nucleic acid comprising a nucleotide sequence encoding the fusion protein;
an expression vector comprising the nucleic acid operatively linked to a control sequence;
a cell comprising the fusion protein, the nucleic acid, or the expression vector; and/or
a pharmaceutical composition comprising the fusion protein, the nucleic acid, the expression vector, or the cell, thereby treating the condition in the subject.

84-85. (canceled)

86. A method of protecting ocular photoreceptor cells in a subject in need thereof, comprising administering to an eye of the subject an effective amount of the fusion protein of claim 1;
a nucleic acid comprising a nucleotide sequence encoding the fusion protein;
an expression vector comprising the nucleic acid operatively linked to a control sequence;
a cell comprising the fusion protein, the nucleic acid, or the expression vector; and/or
a pharmaceutical composition comprising the fusion protein, the nucleic acid, the expression vector, or the cell, thereby protecting the ocular photoreceptor cells in the subject.

87-88. (canceled)

89. A method for producing a fusion protein of claim 1, comprising culturing cell comprising the fusion protein;
a nucleic acid comprising a nucleotide sequence encoding the fusion protein; or
an expression vector comprising the nucleic acid operatively linked to a control sequence, under conditions allowing for expression and secretion of the encoded fusion protein, and isolating the fusion protein from the cell culture.

90. A fusion protein comprising a first N-terminal signal peptide, a second peptide C-terminal to the signal peptide, and a third peptide C-terminal to the second peptide;
wherein the first peptide is a human immunoglobulin kappa chain (Igk) signal sequence; and
wherein the second peptide or the third peptide is an RdCVF-short peptide and the other is a hydrophilic peptide.

91-127. (canceled)

128. A nucleic acid comprising a nucleotide sequence encoding a fusion protein of claim 90.

129-133. (canceled)

134. An expression vector comprising the nucleic acid of claim 128, operatively linked to a control sequence.

135-142. (canceled)

143. A cell comprising the fusion protein of claim 90; a nucleic acid comprising a nucleotide sequence encoding the fusion protein; or an expression vector encoding the nucleic acid operatively linked to a control sequence.

144. A pharmaceutical composition comprising: (i) the fusion protein of claim 90;
a nucleic acid comprising a nucleotide sequence encoding the fusion protein;
an expression vector comprising the nucleic acid operatively linked to a control sequence; or
a cell comprising the fusion protein, the nucleic acid, or the expression vector;
and (ii) a pharmaceutically acceptable carrier.

145. A method for treating a condition in a mammalian subject in need thereof, comprising administering to the subject an effective amount of the fusion protein of claim 90;
a nucleic acid comprising a nucleotide sequence encoding the fusion protein;
an expression vector comprising the nucleic acid operatively linked to a control sequence;
a cell comprising the fusion protein, the nucleic acid, or the expression vector; and/or
a pharmaceutical composition comprising the fusion protein, the nucleic acid, the expression vector, or the cell,
thereby treating the condition in the subject.

146-147. (canceled)

148. A method of protecting ocular photoreceptor cells in a subject in need thereof, comprising administering to an eye of the subject an effective amount of the fusion protein of claim 90 a nucleic acid comprising a nucleotide sequence encoding the fusion protein;
an expression vector comprising the nucleic acid operatively linked to a control sequence;
a cell comprising the fusion protein, the nucleic acid, or the expression vector; and/or
a pharmaceutical composition comprising the fusion protein, the nucleic acid, the expression vector, or the cell, thereby protecting the ocular photoreceptor cells in the subject.

149-150. (canceled)

151. A method for producing a fusion protein of claim 90, comprising culturing a cell comprising the fusion protein; a nucleic acid comprising a nucleotide sequence encoding the fusion protein: or an expression vector comprising the nucleic acid operatively linked to a control sequence, under conditions allowing for expression and secretion of the encoded fusion protein, and isolating the fusion protein from the cell culture.
